(19) United States
(12) Patent Application Publication
(10) Pub. No.: US 2018/0085054 A1
(43) Pub. Date: Mar. 29, 2018

(54) SLEEP SURFACE SYSTEM AND METHOD THEREOF

(71) Applicants: Scott D. Miles, Sandy, UT (US); Kent F. Beck, Layton, UT (US); Dana L. Miles, Sandy, UT (US)

(72) Inventors: Scott D. Miles, Sandy, UT (US); Kent F. Beck, Layton, UT (US); Dana L. Miles, Sandy, UT (US)

(21) Appl. No.: 15/721,611

(22) Filed: Sep. 29, 2017

Related U.S. Application Data
(63) Continuation-in-part of application No. 14/985,380, filed on Dec. 30, 2015.
Application No. 14/985,380 is a continuation of application No. 13/907,781, filed on May 31, 2013, now Pat. No. 9,247,831.
(60) Provisional application No. 62/401,838, filed on Sep. 29, 2016.
(60) Provisional application No. 61/654,678, filed on Jun. 1, 2012.

(51) Int. Cl. A61B 5/00; A47D 15/00; A47C 21/04; A61B 5/0205; A47C 7/74; A47D 7/00; A47D 9/00

(52) U.S. Cl. CPC A61B 5/4818; A47D 15/001; A47C 21/04; A47C 21/044; A61B 5/6892; A61B 2562/0247; A61B 5/0205; A47C 7/744; A47D 7/00; A47D 9/00; A61B 2503/04; A61B 5/4836

(57) ABSTRACT

A sleep surface system and method for positioning under, for example, an infant or person on a sleeping surface. The sleep surface system includes an insert and a forced air device. The insert includes a top liner and a bottom liner with a space therebetween such that the top liner defines pores therein. In addition, the forced air device is configured to provide air flow through the pores and adjacent a person or infant.

### CROSS-REFERENCE TO RELATED APPLICATIONS

This application claims the benefit of U.S. Provisional Application No. 62/401,838, filed Sep. 29, 2016, the disclosure of which is hereby incorporated by reference in its entirety. Further, the present application is a continuation-in-part of U.S. patent application Ser. No. 14/985,380, filed Dec. 30, 2015, which is a continuation of U.S. patent application Ser. No. 13/907,781, filed May 31, 2013, now U.S. Pat. No. 9,247,831, which claims the benefit of U.S. Provisional Application No. 61/654,678, filed Jun. 1, 2012, the disclosures of which are each incorporated by reference herein in their entireties.

### TECHNICAL FIELD

The present invention relates generally to a surface for resting or sleeping and, more specifically, the present invention relates to a sleep surface system and method thereof.

### BACKGROUND

Each year, thousands of infants (aged 2 weeks to 1 year) die from Sudden Infant Death Syndrome (“SIDS”), a disorder in which otherwise healthy infants seemingly stop breathing. Although scientific and medical research has uncovered factors which indicate a predisposition to the disorder (e.g., low birth weight, age of mothers), no specific cause has been uncovered. Moreover, there are varied theories put forth by the medical community as to the cause of SIDS. Some theories suggest a neurological disorder in the infants which intercepts the breathing functions while sleeping and leads to the infant's death by asphyxiation.

Applicant and the medical community believes a contributing cause of SIDS is that infants fall victim to asphyxiation from carbon dioxide re-breathing. More specifically, infants sleeping face down or in close proximity to soft or porous bedding may re-breathe the carbon dioxide in the exhaled air trapped in the air pocket of their bedding near their air passages. Doctors, nurses and medical journals have for years recommended placing an infant on its back for sleeping to avoid this concern. However, many parents and caregivers are reluctant to follow this advice. Some infants prefer sleeping on their stomachs, and do not adjust to the changed position well. Moreover after 5-6 months, most infants can roll themselves over to their preferred sleeping position. Further, many infants have a tendency to regurgitate and parents are concerned their infant may choke on the regurgitated matter.

Based on the foregoing, it would be advantageous to provide a sleep surface that will substantially prevent the conditions which can lead to carbon dioxide poisoning to infants as they sleep.

### BRIEF SUMMARY OF THE INVENTION

The present invention is directed to various embodiments of a sleep surface system. For example, the sleep surface system may be used for positioning under an infant on a sleep surface. In one embodiment, the sleep surface system includes a removable insert and a forced air device. The removable insert is configured to be removably positioned over the sleep surface, the removable insert including a port opening extending to a substantially enclosed space defined between a top liner and a bottom liner with a peripheral boundary defining a coupling between the top and bottom liners. The top liner includes an exterior surface and is formed of a material with pores defined therein such that the pores extend between the enclosed space and the exterior surface of the top liner. Further, the top liner is coupled to the bottom liner at multiple discrete locations positioned separate from the peripheral boundary between the top liner and the bottom liner. The forced air device is configured to provide air flow into the enclosed space defined in the removable insert. With this arrangement, the air flow configured to flow into the enclosed space defined within the removable insert and out of the pores defined therein.

In another embodiment, the removable insert includes a peripheral portion extending from the peripheral boundary sized and configured to extend to an underside of the sleep surface. In another embodiment, the insert includes an elastic portion sized and configured to temporarily couple the insert to the sleep surface.

In still another embodiment, the port opening is configured to facilitate the air flow from the forced air device to the substantially enclosed space defined in the removable insert. In another embodiment, the sleep surface system further includes an elongated plenum configured to be positioned adjacent the peripheral boundary between the top and bottom liners and within the space of the removable insert, the plenum including openings along a length of the elongated plenum for distributing air within the enclosed space of the removable insert. In another embodiment, the bottom liner is a non-porous material.

In accordance with another embodiment of the present invention, a method of flowing air to a face of an infant on an infant sleep surface is provided. The method includes the steps of: positioning a removable insert over a mattress sized for the infant sleep surface, the removable insert including a port opening extending to a substantially enclosed space defined between a top liner and a bottom liner with a peripheral boundary defining a coupling between the top and bottom liners, the top liner formed of a material defining pores therein, the pores extending between an exterior surface and the enclosed space, the top liner being coupled to the bottom liner at multiple discrete locations positioned separate from the peripheral boundary between the top and bottom liners; and forcing air from a forced air device through the port opening and into the enclosed space defined in the removable insert such that the air moves through the pores of the top liner to move air to the face of the infant.

In another embodiment, the method step of positioning includes positioning the removable insert over the infant sleep surface so that a peripheral portion extending from the peripheral boundary of the insert extends to an underside of the sleep surface. In another embodiment, the method step of positioning includes temporarily coupling the insert to the sleep surface with an elastic portion extending from or along the insert. In still another embodiment, the method step of forcing includes distributing the air through an elongated plenum positionable adjacent the peripheral boundary between the top and bottom liners and within the space of the removable insert.

In accordance with another embodiment of the present invention, a sleep surface system for use with a head pillow is provided. The sleep surface system includes a pillow case member, a forced air device, and an elongated plenum. The pillow case member includes a liner with an inner surface and an outer surface, the pillow case member defining an enclosable space within the pillow case member such that the space is sized and configured to receive the head pillow. The pillow case member includes one side end configured to be positioned between an open position and a closed position, the pillow case member including a port opening defined therein. Further, the pillow case member includes a material defining multiple pores therein. The forced air device is configured to provide air flow into the enclosable space defined in the pillow case member through the channel port of the pillow case member, the air flow configured to flow through the port opening and into the enclosable space and out of the multiple pores defined in the liner. The elongated plenum is coupled to the forced air device and positionable adjacent a periphery of the pillow case member and within the space defined in the pillow case member. The elongated plenum includes openings along a length of the elongated plenum and is configured to distribute air within the space of the pillow case member.

In another embodiment, the liner includes an upper liner and a lower liner with the spaced defined therebetween, the upper liner including the multiple pores defined therein to facilitate air flow therethrough. In another embodiment, the lower liner includes a permeable material so as to facilitate air flow through pores of the lower liner.

In another embodiment, the plenum includes a tubular structure. In still another embodiment, the plenum includes a foam material. In another embodiment, the plenum includes one end coupled to an output housing of the forced air device. In another embodiment, the forced air device is positionable at least partially within the port opening of the pillow case member.

In accordance with another embodiment of the present invention, a method of flowing air to a face of a person associated with a head pillow on a sleep surface is provided. The method includes the steps of positioning a pillow case member on the sleep surface, the pillow case member defining an enclosable space within the pillow case member such that the enclosable space is sized and configured to receive the head pillow, the pillow case member including one side end configured to be positioned between an open position and a closed position, the liner of the pillow case member defining a port opening therein, the pillow case member including a material defining multiple pores therein; and forcing air from a forced air device through the port opening of the pillow case member and through an elongated plenum positioned adjacent a periphery of the pillow case member and within the space defined in the pillow case member such that openings defined along a length of the elongated plenum distributes air within the enclosable space within the pillow case member so that the air then moves through the pores of the liner.

In another embodiment, the method step of forcing includes distributing air within the space of the pillow case member with the plenum coupled to an output of the forced air device. In still another embodiment, the method step of positioning includes positioning the pillow case member with an upper liner and a lower liner that collectively substantially defines the enclosable space within the pillow case member such that the upper and lower liners define the pores therein to facilitate air flow therethrough. In another embodiment, the method step of forcing includes filtering the air being forced into the enclosable space of the pillow case member. In still another embodiment, the method step of forcing includes the step of cooling the air being forced into the enclosable space of the pillow case member.

In accordance with another embodiment of the present invention, a blanket system for positioning over a person is provided. The blanket system includes a blanket member and a forced air device. The blanket member is configured to be removably positioned over the person. The blanket member including a top liner and a bottom liner and a port opening, the top liner and the bottom liner define a space therebetween. Further, the top liner is coupled to the bottom liner at multiple discrete locations separate from a periphery of the top and bottom liners, the bottom liner formed of a material with pores defined therein such that the pores extend between the space and an outer surface of the bottom liner so as to facilitate air flow therethrough. The forced air device is configured to provide air flow through the port opening and into the space defined in the blanket member so as to flow air through the pores of the bottom liner.

In another embodiment, the forced air device includes a heating element associated therewith to force heated air through the pores of the blanket member. In another embodiment, the forced air device include a cooling element associated therewith to force cooled air through the pores of the blanket member.

In another embodiment, the blanket system further includes a plenum coupled to the forced air device and positioned adjacent the periphery of the blanket member and within the space of the blanket member. In another embodiment, the plenum includes multiple openings along a length of the plenum to distribute air therethrough. In another embodiment, the blanket member includes batting within the space between the top and bottom liners. In still another embodiment, the top liner includes an impermeable liner.

In accordance with another embodiment of the present invention, a method of flowing air to a person on a sleep surface is provided. The method steps include: positioning a blanket member over the person, the blanket member including a top liner and a bottom liner and a port opening, the top liner and bottom liner defining a space therebetween, the top liner being coupled to the bottom liner at multiple discrete locations separate from a periphery of the top and bottom liners, the bottom liner formed of a material with pores defined therein, the pores extending between the space and an outer surface of the bottom liner so as to facilitate air flow therethrough; and forcing air through the port opening of the blanket member with a forced air device so that the air flows through the pores of the bottom liner.

In another embodiment, the step of forcing includes distributing air within the space with an elongated plenum positioned adjacent the periphery and within the space between the top and bottom liners, the elongated plenum including multiple openings along a length thereof.

In accordance with another embodiment of the present invention, a sleep surface insert system includes a removable insert with a frame, and a forced air device. The removable insert is configured to be removably positioned over the sleep surface. The removable insert includes a top liner having an exterior surface and a bottom liner, the top liner and bottom liner defines at least one space therebetween. The top liner defines multiple pores that extend between the at least one space and the exterior surface. The forced air device is configured to provide air flow into the at least one space defined in the removable insert, the air flow being configured to flow into the removable insert and out of the multiple pores. Further, the removable insert includes a frame, the frame extending adjacent and along a perimeter of the top liner. The frame is sized and configured to move between an expanded first position and a second collapsed position, the expanded first position configured to maintain the top liner in a spread-out horizontal position, the collapsed second position configured to minimize a size of the removable insert.

In one embodiment, upon the frame being in the expanded first position, the frame extends with a continuous single loop. In another embodiment, the frame includes a radial length, a height, and a width, the width being smaller than the height to exhibit a flattened cross-section. In a further embodiment, the flattened cross-section includes at least one of a rectangular periphery and an oval periphery. In another embodiment, upon the frame being moved to the collapsed second position, the frame extends continuously in a multiple loop configuration.

In another embodiment, the forced air device is coupled to the removable insert with a hose. In another embodiment, the frame extends through a padding. In a further embodiment, the padding includes a foam material. In even a further embodiment, the padding includes multiple foam structures positioned adjacent each other along the frame such that the frame is moveable between the expanded first position and the collapsed second position. In another embodiment, the frame is configured to self-expand from a partially collapsed position to the expanded first position.

In another embodiment, the bottom liner of the insert is a non-porous material. In still another embodiment, the bottom liner of the insert is a porous material.

In accordance with another embodiment of the present invention, a sleep surface insert system for positioning under a person on a sleep surface is provided. The sleep surface insert system includes a removable insert, a forced air device, and a gas source. The removable insert is configured to be removably positioned over the sleep surface. Further, the removable insert includes a top liner having an exterior surface and a bottom liner, the top liner and bottom liner defining at least one space therebetween. The top liner defines multiple pores extending between the at least one space and the exterior surface. The forced air device is configured to provide air flow into the at least one space defined in the removable insert, the air flow configured to flow into the removable insert and out of the multiple pores. The gas source is associated with the forced air device such that the gas source is configured to flow a gas with the air flow to flow the gas through the multiple pores.

In one embodiment, the gas includes at least one of oxygen and nitrogen, the oxygen configured to create an oxygen rich atmosphere adjacent the exterior surface of the removable insert and the nitrogen configured to create an oxygen poor atmosphere adjacent the exterior surface of the removable insert. In another embodiment, the removable insert is sized to be associated with a pillow.

In still another embodiment, the removable insert includes a padding positioned within the space of the removable insert. In a further embodiment, the padding is a reticulated foam configured to facilitate air flow therethrough. In another further embodiment, the sleep surface insert system includes a shroud configured to extend at least partially over the insert.

In accordance with another embodiment of the present invention, a sleep surface insert system for positioning under a person on a sleep surface is provided. The sleep surface insert system includes a removable insert and a gas source. The removable insert is configured to be removably positioned over the sleep surface, the removable insert having a top liner having an exterior surface and a bottom liner, the top liner and bottom liner defining at least one space therebetween. The top liner defines multiple pores that extend between the at least one space and the exterior surface. The gas source is configured to provide a gas to flow into the at least one space defined in the removable insert, the gas being configured to flow into the removable insert and out of the multiple pores.

In one embodiment, the gas includes at least one of oxygen and nitrogen, the oxygen configured to create an oxygen rich atmosphere adjacent the exterior surface of the removable insert and the nitrogen configured to create an oxygen poor atmosphere adjacent the exterior surface of the removable insert. In another embodiment, the removable insert includes a padding positioned within the space of the removable insert. In a further embodiment, the padding is a reticulated foam configured to facilitate air flow therethrough. In another embodiment, the sleep surface insert system further includes a shroud configured to extend at least partially over the insert.

In accordance with another embodiment of the present invention, a sleep surface insert system for positioning under a person on a sleep surface is provided. The sleep surface insert system includes a removable insert and a forced air device. The removable insert is configured to be removably positioned over the sleep surface. The removable insert includes a top liner having an exterior surface and a bottom liner, the top liner and bottom liner defining at least one space therebetween. The top liner defines multiple pores extending between the at least one space and the exterior surface. The forced air device is configured to provide air flow from above the exterior surface of the top liner and through the multiple pores and into the at least one space defined in the removable insert, the air flow configured to flow toward and through the forced air device and through a filter.

In one embodiment, the filter is at least one of an absorbent charcoal filter and a molecular sieve. In another embodiment, the removable insert includes a foam material positioned within the space defined in the removable insert, the foam material sized to maintain a gap between the top liner and the bottom liner. In another embodiment, the sleep surface insert system further includes a sensor, the sensor configured to detect levels of an agent from the air flow. In another embodiment, the forced air device is a suction device. In another embodiment, the sleep surface insert system further includes a shroud configured to extend at least partially over the insert.

In accordance with another embodiment of the present invention, a method of removing an agent being exhaled from a person is provided. The method includes the steps of: positioning a removable insert over a sleep surface, the removable insert having a top liner and a bottom liner defining a space therebetween, the top liner defining pores extending between an exterior surface and the space; and suctioning air and the agent from the exterior surface and through the pores and through the space with a suctioning device so that the agent is absorbed by a filter.

In another embodiment, the method further includes the step of sensing the agent with a sensor associated with the suctioning device. In another embodiment, the positioning step includes positioning a removable insert with a padding disposed within the space defined between the top liner and the bottom liner. In another embodiment, the positioning step includes positioning a shroud over the insert.

In accordance with another embodiment of the present invention, a method of flowing a gas to a person is provided. The method includes the steps of: positioning a removable insert over a sleep surface, the removable insert having a top liner and a bottom liner defining a space therebetween, the top liner defining pores extending between an exterior surface and the space; and forcing a gas from a gas source toward the removable insert and through the space defined in the insert such that the gas moves through the pores of the top liner.

In one embodiment, the forcing step includes delivering the gas with a pressurized tank. In another embodiment, the method further includes the step of forcing air toward the removable insert with a blower device such that the gas flows with the air from the blower device through the space of the insert and the pores defined in the top liner. In another embodiment, the positioning step includes positioning a removable insert with a padding disposed within the space defined between the top liner and the bottom liner. In another embodiment, the method further includes the step of sensing a gas content being moved to the exterior surface of the insert. In still another embodiment, the method further includes the step of maintaining the gas moved to the exterior surface of the insert with a shroud.

In accordance with another embodiment of the present invention, a sleep surface insert system for positioning under a person on a sleep surface is provided. The sleep surface insert system includes a removable insert and a forced air system. The removable insert is configured to be removably positioned over the sleep surface. The removable insert includes a top liner having an exterior surface and a bottom liner, the top liner and bottom liner defining at least one space therebetween. The top liner defines multiple pores extending between the at least one space and the exterior surface. Further, the insert includes a padding member positioned between the top liner and the bottom liner, the padding member having a periphery smaller than a periphery of the top liner or the bottom liner. With this arrangement, the forced air system is configured to provide air flow into the at least one space defined in the removable insert such that the air flow is configured to flow into the removable insert and out of the multiple pores.

In one embodiment, the insert is moveable between a use position and a compact position, the use position being a substantially flat spread-out position and the compact position being at least one of a rolled-up position and a folded position. In another embodiment, the insert is moveable between a spread-out and flat use position and a rolled-up compact position.

In another embodiment, the padding member is a flexible soft material. In another embodiment, the padding member is at least one of an elastomer material and a foam material. In still another embodiment, the bottom liner is a porous material.

In another embodiment, the sleep surface insert system further includes a sensor associated with the forced air system, the sensor configured to sense the person positioned on the insert. In another embodiment, the forced air system includes a controller associated with the sensor, the controller configured to turn on or off the forced air system upon the sensor sensing the person on or off the insert. In still another embodiment, the sensor is configured to sense at least one of pressure and temperature.

In accordance with another embodiment of the present invention, a sleep surface insert system for positioning under a person on a sleep surface is provided. The sleep surface insert system includes a removable insert and a forced air system. The removable insert is configured to be removably positioned over the sleep surface, the removable insert including a top liner having an exterior surface and a bottom liner. The top liner and bottom liner defines at least one space therebetween, the top liner defining multiple pores extending between the at least one space and the exterior surface. Further, in this embodiment, the bottom liner includes a padding structure positioned below the top liner, the padding structure having a periphery corresponding with a periphery of the top liner. With this arrangement, the forced air system is configured to provide air flow into the at least one space defined in the removable insert, the air flow configured to flow into the removable insert and out of the multiple pores.

In one embodiment, the insert is moveable between a use position and a compact position, the use position being a substantially flat spread-out position and the compact position being at least one of a rolled-up position and a folded position. In another embodiment, the insert is moveable between a spread-out and flat use position and a rolled-up compact position.

In another embodiment, the padding structure is a flexible soft material. In another embodiment, the padding structure includes at least one of an elastomer material and a foam material.

In another embodiment, the sleep surface insert system further includes a sensor associated with the forced air system, the sensor configured to sense the person positioned on the insert. In another embodiment, the forced air system includes a controller associated with the sensor, the controller configured to turn on or off the forced air system upon the sensor sensing the person on or off the insert. In another embodiment, the sensor is configured to sense at least one of pressure of the person on the insert and a temperature of the person on the insert.

### BRIEF DESCRIPTION OF THE SEVERAL VIEWS OF THE DRAWINGS

The foregoing and other advantages of the invention will become apparent upon reading the following detailed description and upon reference to the drawings in which:

FIG. 1 is a partial schematic view of a sleep surface insert system, depicting an insert and a blower system, according to an embodiment of the present invention;

FIG. 1A is an enlarged view of region A of the insert of FIG. 1, according to one embodiment of the present invention;

FIG. 2 is a cross-sectional view of the insert taken along section B of FIG. 1, according to another embodiment of the present invention;

FIG. 3 is a cross-sectional view of the insert taken along section C of FIG. 2, according to another embodiment of the present invention;

FIG. 4 is another embodiment of a sleep surface insert system, according to another embodiment of the present invention;

FIG. 5 is a perspective view of another embodiment of a sleep surface insert system, depicting an insert having horizontal portion and vertical portion thereof, according to the present invention;

FIG. 5A is a cross-sectional view of the insert taken along section 5A of FIG. 5, according to another embodiment of the present invention;

FIG. 5B is an enlarged view of region 5B of the insert of FIG. 5, according to another embodiment of the present invention;

FIG. 5C is an enlarged view of another embodiment of region 5B of the insert of FIG. 5, according to the present invention;

FIG. 6 is a cross-sectional view of another embodiment of the sleep surface insert system of FIG. 5, depicting a removable pad liner to be positioned in the insert, according to the present invention;

FIG. 7 is a perspective view of another embodiment of an air flow insert system, depicting an insert including a vertical portion, according to the present invention;

FIG. 7A is a cross-sectional view of the insert taken along section 7A of FIG. 7, according to another embodiment of the present invention;

FIG. 7B is an enlarged view of region 7B of the insert of FIG. 7, according to another embodiment of the present invention;

FIG. 8 is a partially schematic view of a sleep surface insert system, depicting a top view of an insert of the sleep surface insert system, according to one embodiment of the present invention;

FIG. 8A is an enlarged cross-sectional view of an a frame of the insert taken along section 8A of FIG. 8, according to another embodiment of the present invention;

FIG. 8B is an enlarged cross-sectional view of another embodiment of a frame of the insert, according to the present invention;

FIG. 8C is an enlarged cross-sectional view of another embodiment of a frame of the insert, according to the present invention;

FIG. 9A is a simplified outline view of a frame of an insert, depicting the frame in a first bent position, according to another embodiment of the present invention;

FIG. 9B is a simplified outline view of the frame, depicting the frame in a further bent position from that depicted in FIG. 9A, according to another embodiment of the present invention;

FIG. 9C is a simplified outline view of the frame, depicting the frame in even a further bent position from that depicted in FIG. 9B, according to another embodiment of the present invention;

FIG. 9D is a simplified outline view of the frame, depicting the frame in a collapsed position, according to another embodiment of the present invention;

FIG. 10 is a top view of a frame of an insert with a padding (without the liners and sleeve surrounding the frame), according to another embodiment of the present invention;

FIG. 10A is an enlarged cross-sectional view of the insert taken along section 10A of FIG. 10, depicting the frame surrounded with the padding and sleeve and the liners, according to another embodiment of the present invention;

FIG. 11 is a partial top view of a frame of an insert with a padding (without liners and sleeve surrounding the frame), according to another embodiment of the present invention;

FIG. 11A is an enlarged cross-sectional view of the insert taken along section 11A of FIG. 11, depicting the frame surrounded with the padding and sleeve and the liners, according to another embodiment of the present invention;

FIG. 11B is an enlarged cross-sectional view of another embodiment of a padding of the insert, according to the present invention;

FIG. 12 is a partial top view of a frame of an insert exhibiting elongated structures (without liners and sleeve surrounding the frame), according to another embodiment of the present invention;

FIG. 12A is an enlarged cross-sectional view of the insert taken along section 12A of FIG. 12, depicting the frame with the sleeve and liners, according to another embodiment of the present invention;

FIG. 12B is an enlarged cross-sectional view of another embodiment of a frame of the insert, according to the present invention;

FIG. 12C is an enlarged cross-sectional view of another embodiment of a frame of the insert, according to the present invention;

FIG. 12D is an enlarged cross-sectional view of another embodiment of a frame of the insert, according to the present invention;

FIG. 13 is a partial schematic view of a sleep surface insert system, depicting a perspective view of the insert, according to another embodiment of the present invention;

FIG. 13A is a cross-sectional view of the insert of the sleep surface insert system taken along section 13A of FIG. 13, according to another embodiment of the present invention;

FIG. 13B is a cross-sectional view of another embodiment of an insert of the sleep surface insert system, according to the present invention;

FIG. 13C is a cross-sectional view of another embodiment of an insert of the sleep surface insert system, according to the present invention;

FIG. 14 is a partial schematic view of a sleep surface insert system, depicting a perspective view of an insert, according to another embodiment of the present invention;

FIG. 14A is a cross-sectional view of the insert of the sleep surface insert system taken along section 14A of FIG. 14, according to another embodiment of the present invention;

FIG. 14B is a cross-sectional view of another embodiment of an insert of the sleep surface insert system, according to the present invention;

FIG. 15 is a partial perspective view of a sleep surface insert system, depicting an insert with a shroud positioned therewith, according to another embodiment of the present invention;

FIG. 16 is a perspective view of an insert of a sleep surface insert system, according to another embodiment of the present invention;

FIG. 17 is a simplistic cross-sectional view of a sleep surface insert system, depicting a forced air system disposed internal an insert, according to another embodiment of the present invention;

FIG. 18 is a perspective view of an insert of a sleep surface insert system, according to another embodiment of the present invention;

FIG. 18A is a cross-sectional view of the insert taken along section line 18A of FIG. 18, depicting a padding structure disposed between a top liner and bottom liner of the insert, according to another embodiment of the present invention;

FIG. 18B is a cross-sectional view of another embodiment of the insert of a sleep surface insert system, according to the present invention;

FIG. 19 is a side view of an insert of a sleep surface insert system, depicting the insert in a compact position, according to another embodiment of the present invention;

FIG. 20 is a perspective view of a sleep surface system for positioning over an infant's mattress, depicting a forced air device in schematic form, according to another embodiment of the present invention;

FIG. 20A is a cross-sectional side view of the sleep surface system taken along section A-A in FIG. 20, depicting air flow from a top surface of an insert of the sleep surface system, according to another embodiment of the present invention;

FIG. 20B is a cross-sectional view of the sleep surface system taken along section B-B in FIG. 20, depicting air flow from a top surface of an insert of the sleep surface system along multiple discrete attachment locations, according to another embodiment of the present invention;

FIG. 21 is a perspective view of another embodiment of a sleep surface system, depicting a pillow case member with a forced air device, according to the present invention;

FIG. 22 is a perspective view of a plenum and a forced air device, according to another embodiment of the present invention;

FIG. 22A is a cross-sectional view of the plenum taken along section A-A of FIG. 22, according to another embodiment of the present invention;

FIG. 23 is a side view of the sleep surface system, depicting air flow moving generally upward from an outer liner of the sleep surface system, according to another embodiment of the present invention;

FIG. 24 is a perspective view of another embodiment of a sleep surface system, depicting a blanket member and a forced air device, according to the present invention;

FIG. 24A is a cross-sectional side view of the sleep surface system taken along section A-A of FIG. 24, depicting a plenum positioned within the blanket member and air flow moving generally downward from the blanket member, according to another embodiment of the present invention; and

FIG. 25 is a partial bottom view of the blanket member, depicting a woven material of the bottom liner, according to another embodiment of the present invention.

### DETAILED DESCRIPTION OF THE INVENTION

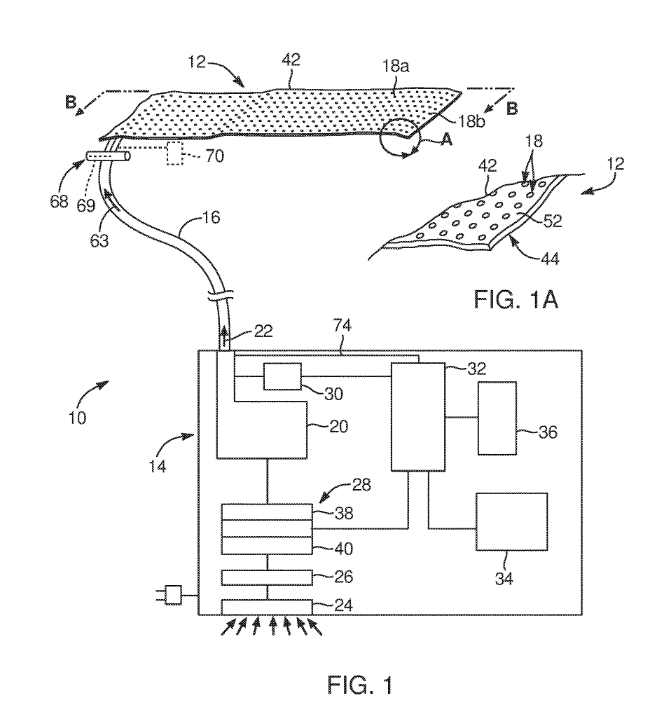

Referring to FIG. 1, a sleep surface insert system 10 configured to, for example, substantially eliminate the conditions leading to SIDS is provided. As will be apparent to one of ordinary skill in the art, the insert system may be employed for other purposes, as set forth herein in the various embodiments. In one embodiment, such a sleep surface insert system 10 may provide oxygen to an infant (not shown) positioned over a portion of the system 10 while also jostling or slightly rocking the infant lying thereon. In another embodiment, the sleep surface insert system 10 may monitor the heart rate and/or respiration of the infant while providing oxygen to an infant positioned over a portion of the system 10. For example, the sleep surface insert system 10 may include an insert 12 and a blower system 14 with a conduit 16 extending therebetween. The blower system 14 may be configured to direct air flow through the conduit 16 and into the insert 12 to inflate portions of the insert 12, in an alternating manner, to slightly rock or jostle an infant positioned over the insert 12 while also flowing oxygen through multiple pores 18 in the insert, thereby, ensuring ample oxygen is present for the infant. In this manner, such movement of the infant may ensure the elimination of potential air pockets of carbon dioxide forming while also providing oxygen through the multiple pores 18. Further, such movement of the infant may gently stimulate the infant's nervous system, maintaining it in a state to properly function or respond to external or internal stimuli or conditions. In addition, the insert 12 may be a substantially flat configuration such that it may be positioned over a mattress (not shown) or other suitable sleeping surface. Further, the insert 12 may include a comfort liner (not shown) that may be slipped over the insert 12 as well as removed to wash when necessary.

The blower system 14 may include a blower 20 configured to blow air, as indicated by arrow 22, into the insert 12 via the conduit 16. Further, the blower system 14 may include an air intake 24, a filter 26, a thermal element 28, one or more sensors 30, a controller 32, a display 34, and a manual control 36. The blower 20 may pull air from the air intake 24 toward the blower 20 and then push the air through the conduit 16 and into the insert 12. In addition, the filter 26 may be provided between the conduit 16 and the air intake 24 and, more particularly, the filter 26 may be positioned between the air intake 24 and the blower 20. The thermal element 28 may also be positioned between the air intake 24 and the conduit 16. The controller 32 may be coupled to the one or more sensors 30 and the thermal element 28 and, further, the manual control 36. Also, the controller 32 may be coupled to the display 34. The one or more sensors 30 may be positioned along an air flow path to sense air temperature and/or air pressure.

In one embodiment, air temperature of the sleep surface insert system 10 may be controlled. For example, the air temperature may be controlled by manually setting a desired temperature for an infant via the manual control 36. The one or more sensors 30 may sense the actual temperature of the air and then relay the actual temperature to the controller 32. The controller 32 may then control the thermal element 28, positioned in an air flow path, to modify the air temperature from the actual air temperature to the desired air temperature.

In one embodiment, the thermal element 28 may be positioned in the air flow path between the air intake 24 and the blower 20. In another embodiment, the thermal element 28 may be positioned in the air flow path between the blower 20 and the conduit 16 or, otherwise said, an external portion of the conduit 16. In one embodiment, the thermal element 28 may include a heating element 38. In another embodiment, the thermal element 28 may include a cooling element 40. In still another embodiment, the thermal element 28 may include both a heating element 38 and a cooling element 40. Such a heating element 38 and cooling element 40 may be directly coupled adjacent to each other or separate from each other. The heating element 38 may be a coil or the like or any other suitable heating element known in the art, such as a peltier element employed as a heating element. The cooling element 40 may be a peltier element or any other suitable cooling element known in the art.

With respect to FIGS. 1, 2, and 3, the insert 12 may include a top liner 42 and a bottom liner 44 with one or more hollow spaces defined therebetween. In one embodiment, a single hollow space (not shown) may be defined between the top liner 42 and the bottom liner 44. In another embodiment, the insert 12 may include a lateral wall 42 extending transverse or substantially orthogonal between and relative to the top liner 42 and the bottom liner 44. Such lateral wall 46 may separate and define a first hollow space 48 and a second hollow space 50 between the top liner 42 and the bottom liner 44. In another embodiment, the lateral wall 46 may define multiple hollow spaces between the top liner 42 and the bottom liner 44, for example, three or more hollow spaces.

With reference to FIGS. 1, 1A, and 3, the top liner 42 may include an outer surface 52, or exterior surface, and an inner surface 54 with a periphery defining an edge 56 of the top liner 42. The bottom liner 44 may include a bottom surface 58 and an inner surface 60 defined by a bottom liner periphery 62. The top liner 42 may be sized and configured to substantially correspond with dimensions of the bottom liner 44 such that their respective peripheries may be coupled together or, rather, the portion adjacent their respective peripheries may be coupled together.

Further, the top liner 42 may include multiple pores 18 extending between the outer surface 52 and the inner surface 54 configured to facilitate air flow therethrough. Such multiple pores 18 may be in the form of holes, as depicted in FIG. 1A, formed through the top liner 42 extending between the first and second hollow spaced 48, 50 and the outer surface 52 of the top liner 42. The multiple pores 18 may include first multiple pores 18a and second multiple pores 18b that may be distinguished by corresponding with the respective first and second hollow spaces 48, 50 defined in the insert 12.

The multiple pores 18 may be in the form of an array such that the multiple pores 18 may be sized, positioned and/or spaced in a predetermined manner. In this embodiment, the top liner 42 may include a polymeric material in a sheet like form. In another embodiment, the multiple pores 18 may be randomly sized, positioned and/or spaced. In this embodiment, the top liner 42 may be formed from a weaved material or the like such that the multiple pores 18 are inherent to the gaps or spaces formed in the weaved material. Such a top liner 42 may be formed from a synthetic material, such as a polymeric material, or the top liner may be formed from a natural material, such as a cotton, or various combinations or blends thereof or any other suitable material that is safe and provides multiple pores 18 so as to facilitate air through the multiple pores 18, as known by one of ordinary skill in the art. The bottom liner 44 may be formed of a solid sheet like material, without pores, so as not to facilitate air to flow therethrough. In another embodiment, the bottom liner 44 may be formed to include pores, similar to the top liner 42. Further, in one embodiment, the top liner 42 and the bottom liner 44 may be coupled together via heat pressing, sewing or any other known or suitable method of coupling. In another embodiment, the top liner 42 and the bottom liner 44 may be formed of a continuous, unitary, and/or seamless material.

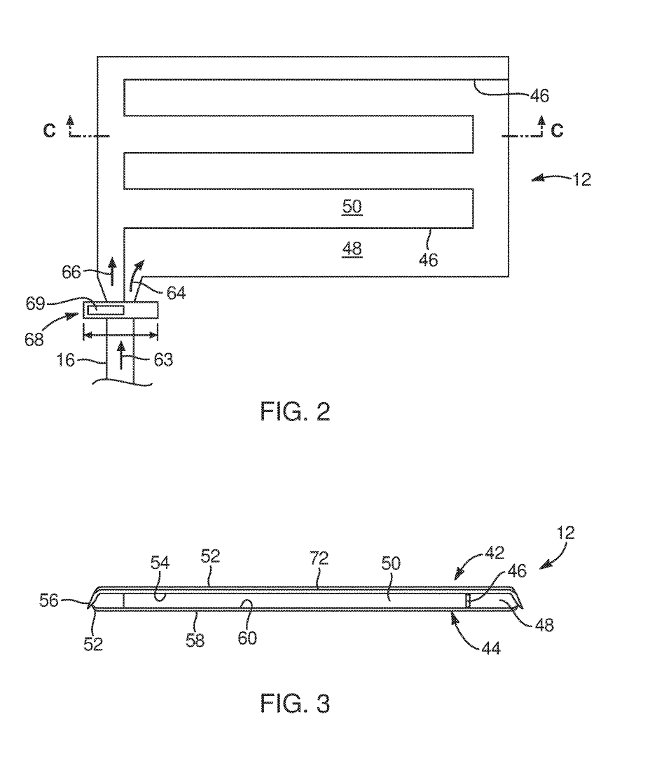

Referring now to FIGS. 1 and 2, the conduit 16 may be coupled between the blower 20 or blower system 14 and the insert 12. Further, the conduit 16 may branch from a single flow path 63 to a first flow path 64 and a second flow path 66, the first flow 64 path configured to correspond with the first hollow space 48 and the second flow path 66 to correspond with the second hollow space 50 defined in the insert 12. Further, the conduit 16 may include an actuator 68 coupled thereto. The actuator 68 may be sized and configured to alternate air flow in the conduit 16 between the first flow path 64 and the second flow path 66, thereby, alternating air flow between the first hollow space 48 and the second hollow space 50 defined in the insert 12. With this arrangement, the actuator 68 alternates air flow between the first hollow space 48 and the second hollow space 50 defined in the insert 12, thereby, facilitating movement of the insert 12 so that an infant lying over the insert 12 may continuously be rocked or lightly jostled to, for example, gently stimulate the infant's nervous system to encourage proper function and reaction to diminished oxygen or, rather, excessive carbon dioxide. In addition, air flowing through the multiple pores 18 further ensures ample oxygen for the infant that may be lying face down on a sleep surface.

The actuator 68 may be a timed actuator in the form of a solenoid 69. For example, the actuator 68 may actuate at a frequency. Such a frequency may be manually modified to control the cycle by which air flow is directed into the first and second hollow spaces 48, 50 for alternately inflating different portions of the insert 12. The actuator 68 may also include a pressure sensor 70 that may be configured to actuate upon sensing a predetermined air pressure within the first flow path 64 and the second flow path 66 and, thus, the air pressure within the respective first and second hollow spaces 48, 50 defined in the insert 12. In another embodiment, the actuator 68 may be a bladder controlled diverter (not shown). Such a bladder controlled diverter may be sized and configured to inherently and mechanically actuate between a first position and a second position upon the respective first hollow space 48 and the second hollow space 50 of the insert 12 or bladder inflating to a predetermined air pressure. In other words, the bladder controlled diverter may automatically actuate between the first and second positions upon portions of the bladder, i.e., first and second hollow spaces 48, 50 in the insert, reaching predetermined inflation levels.

Referring now to FIGS. 1 and 3, in another embodiment, as previously set forth, the sleep surface insert system 10 may monitor various physiological parameters of the infant (or person), such as, the heart rate and/or respiration of an infant while also providing oxygen to the infant positioned over the insert 12 of the system 10. Other physiological parameters may include movement, moisture, such as bed wetting, and a temperature of the infant. Such monitoring may be employed with one or more sensors, such as, a pressure sensor, conductive polymer (resistance inflections), closely separated conductors, i.e., capacitance/resistance inflections. For example, the insert 12 may include a sensor in the form of a piezoelectric film 72. Such film may include lines or wires 74 extending from the insert 12 along the conduit 16 to be coupled to, for example, the controller, which may then provide information at the display 34. The piezoelectric film 72 may be configured to sense slight force, movement, thermal, and/or pressure changes to facilitate monitoring an infant's heart rate and respiration. The controller 32 may also be configured to provide an alarm (not shown) if significant changes in the heart rate and respiration are sensed to ensure potential issues with an infant are alleviated. Such alarm may be set-off from, for example, the system itself or transmitted remotely to a location of the infant's parents.

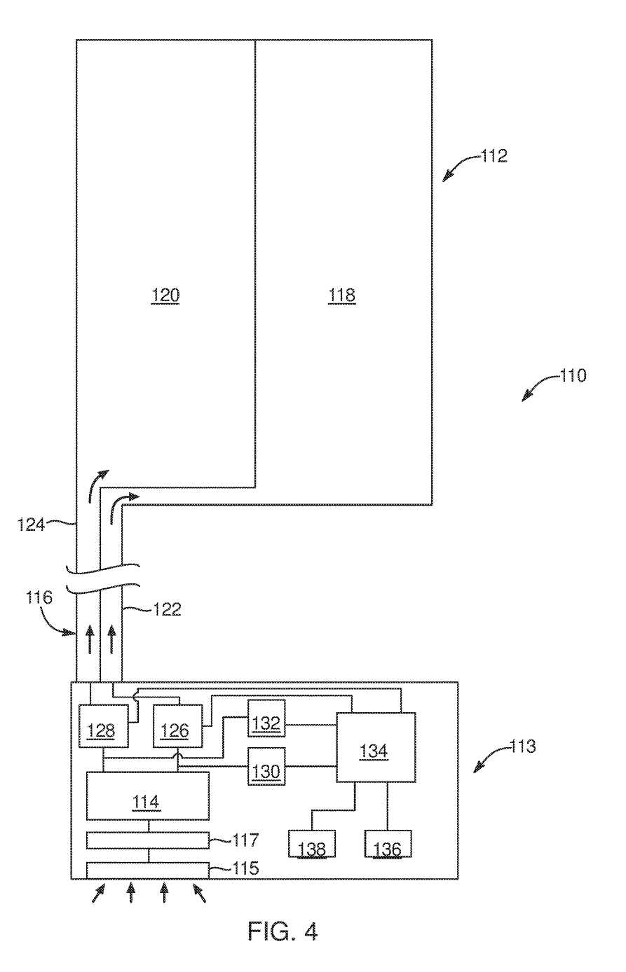

Now referring to FIG. 4, a sleep surface insert system 110 of another embodiment is provided. This system is similar in many respects to the previous embodiment of FIG. 1, however, the sleep surface insert system 110 of this embodiment may not include an actuator to alternate air flow between two hollow spaces defined in an insert. Rather, in this embodiment, one primary function of the sleep surface insert system 110 may be configured to control temperature of air being blown through pores (not shown) defined in the top liner of the insert 112.

For example, the sleep surface insert system 110 may include an insert 112 and a blower system 113 with conduit 116 extending therebetween. The blower system 113 may include an air intake 115 and filter 117, as set forth in the previous embodiment. In one embodiment, the insert 112 may include a first hollow space 118 and a second hollow space 120 defined in the insert 112 between a top liner and a bottom liner (not shown). Each hollow space defined in the insert 112 may include its own conduit extending from the insert to the blower system, namely, a first conduit 122 and a second conduit 124. The first and second conduits 122, 124 may be in the form of a single elongated flexible member, but include respective first and second lumens to channel air flow to the respective first and second hollow spaces 118, 120 defined in the insert 112. The blower system 113 may include a single blower 114 or two blowers dedicated to the respective first and second conduits 122, 124. Further, each air flow path for the first and second conduits 122, 124 may include a first thermal element 126 and a second thermal element 128, respectively. Each thermal element may each include a cooling element and a heating element. The blower system 113 may also include the other components necessary to control a temperature of the air being blown toward the insert 112. For example, the blower system 113 may include a first sensor 130 and a second sensor 132 configured to sense the actual temperature being blown and a controller 134 coupled to the first and second thermal elements 126, 128 to modify the actual temperature to a desired temperature as manually set via a first and second manual control 136, 138. With this arrangement, the sleep surface insert system 110 may be configured to control the temperature over two sleep surface portions of the insert 112 such that the defined first and second hollow spaces 118, 120 correspond with sleep surface portions typical for two persons lying next to each other. Such a system 110 may be desirable for couples who desire different temperatures for their respective sleep portions. Further, the insert 112 may be sized and configured for single person users such that the insert 112 may include a single hollow space with a corresponding single thermal element and sensor in the blower system.

In one embodiment, the insert 112 may be sized and configured to be positioned over a pillow or within a pillow cover. In another embodiment, the insert 112 may be sized and configured to act as a pillow itself while employing one or various combinations of the functions described herein with the sleep surface insert system 110, such as monitoring temperature and temperature control, air filtering, inflation of one hollow space defined in an insert, and alternating (or sequencing) inflation of two or more hollow spaced defined within an insert. In still another embodiment, by employing various functions described herein, the sleep surface insert system 10, 110 may be employed to monitor not only infants, but to monitor sleep disorders, such as snoring, sleep apnea or any other suitable sleep disorder. In another embodiment, the insert 10 described in FIG. 1 may be sized to act with or as a pillow and include a single chamber (or multiple chambers) defined in the insert. Such insert of this embodiment may include various controls and/or sensors to assist in monitoring, for example, sleep apnea, asthma, or a temperature of the person laying on the pillow with the insert, and utilize one or more of the functions described herein, such as filtering the air flowing into the insert, controlling the temperature of the air, and/or re-orienting the user's head to discourage snoring.

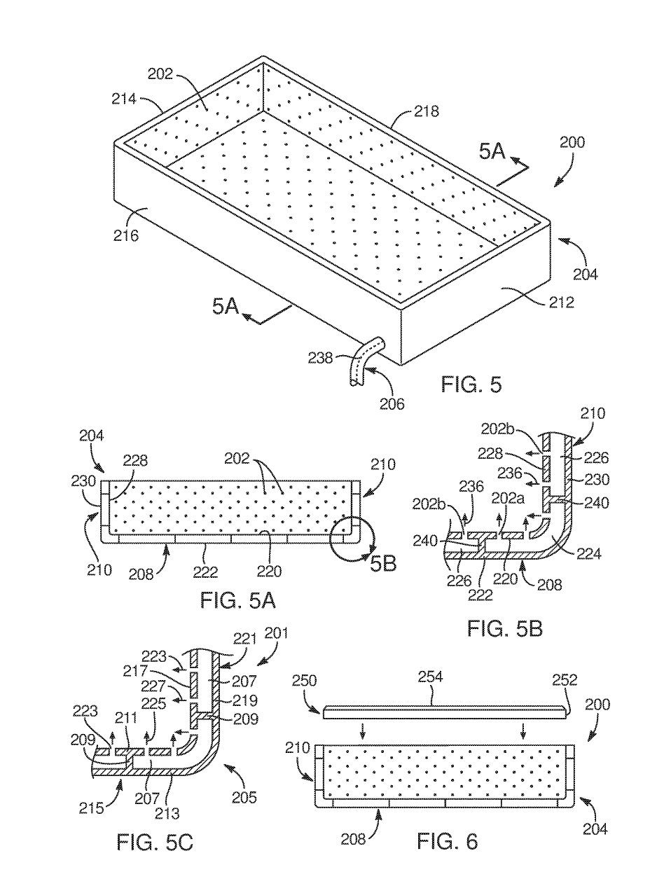

Now referring to FIG. 5, another embodiment of a sleep surface insert system 200 is provided. In this embodiment, the sleep surface insert system 200 may be similar to the embodiment described in FIGS. 1 through 3, but may also include transversely or vertically extending and upstanding side walls, as depicted, that provide a wall or barrier to the sleep surface insert system 200. The sleep surface insert system 200 may be sized and configured to be positioned over a crib-sized mattress (not shown) or positioned over any sleep surface so as to provide air flow through apertures 202 or pores, similar to that previously described. The vertical side walls also provide cushioning and a barrier for a baby's head and body from the rigid bars or walls of, for example, an infant's crib. In another embodiment, the side walls may be transversely angled or include a radius, but still include a vertical component extending above the insert 204.

With respect to FIGS. 5, 5A, and 5B, the sleep surface insert system may include an insert 204, a blower system 14 (FIG. 1), and conduit 206 extending between the insert 204 and the blower system 14. The insert 204 may include a first portion or horizontal portion 208 and a second portion or vertical portion 210, the vertical portion 210 extending transverse or upward from a periphery of the horizontal portion 208 so as to provide the wall or barrier. It is noted that the vertical portion 210 is not necessarily precisely vertical relative to a horizontal plane, but rather, may include a vertical component that is transverse relative to the horizontal portion of the insert 204 to provide the wall or barrier.

In one embodiment, for example, the horizontal portion 208 may be rectangular and substantially flat such that the vertical portion 210 may include first and second facing vertical sides 212, 214 and third and fourth facing vertical sides 216, 218, each of which may extend upward and transverse relative to the horizontal portion 208. In one embodiment, the various sides of the vertical portion 210 may extend generally or substantially vertical relative to the horizontal portion 208 with the transition from the horizontal portion 208 to the vertical portion 210 being arcuate. The first vertical side 212 may be contiguous with the third and fourth vertical sides 216, 218. Likewise, the second vertical side 214 may be contiguous with the third and fourth vertical sides 216, 218. The periphery of the horizontal portion 208 of the insert 204 may include other shapes, such as oval or any other suitable shape, such that the vertical portion extends upward from the periphery of the horizontal portion to provide the wall or barrier for an infant.

The horizontal portion 208 may include a top liner 220 and a bottom liner 222 that define first and second hollow spaces 224, 226 or chambers therebetween, similar to the insert 12 depicted in FIGS. 1 through 3, but the first and second hollow spaces 224, 226 may continue into the vertical portion 210 of the insert 204. The vertical portion 210 may include an inner liner 228 and an outer liner 230 also defining the first and second hollow spaces 224, 226 or chambers. The inner liner 228 may be contiguous with the top liner 220 Likewise, the bottom liner 222 may be contiguous with the outer liner 230 of the insert.

The top liner 220 of the horizontal portion 208 and the inner liner 228 of the vertical portion 210 may include apertures 202 defined therein. For example, the apertures 202 defined in the top liner 220 and the inner liner 228 may each include first apertures 202a and second apertures 202b, the first apertures 202a corresponding with the first hollow space 224 and the second apertures 202b corresponding with the second hollow space 226. Similar to the horizontal portion 208, each of the vertical sides of the vertical portion 210 may each include one of the first and second hollow spaces 224, 226, or both. Further, the first and second hollow spaces 224, 226 may be separated by one or more impermeable lateral connections 240. Such lateral connections 240 assist in maintaining the top and bottom liners 220, 222 of the horizontal portion 208 in a generally parallel configuration Likewise, the lateral connections 240 assist in maintaining the inner liner 228 generally parallel relative to the outer liner 230 of the vertical portion 210. Further, the lateral connections 240 may serve to separate the first and second hollow spaces 224, 226 in a discrete and distinct manner.

The blower system 14 (FIG. 1) may provide air pressure or air flow 236 into each of the first and second hollow spaces 224, 226 such that air flow 236 moves outward from the apertures 202 associated with each of the first and second hollow spaces 224, 226. As depicted in FIG. 5B, the air flow 236 moves generally in a horizontal direction (as depicted with horizontal direction arrows) from the vertical portion 210 of the insert 204 and generally in a vertical direction (as depicted with vertical direction arrows) from the horizontal portion 208 of the insert 204. It is noted that the vertical portion 210 may not extend precisely vertical relative to the horizontal portion 208, but rather, may extend upward and transverse relative to the horizontal portion 208 so as to provide a wall or barrier. As such, the apertures 202 in the vertical portion 210 may provide air flow in a general horizontal direction such that the air flow 236 flowing from the vertical portion 210 flows transverse relative the air flow 236 flowing from the horizontal portion 208. In this manner, the vertical portion 210 provides the wall or barrier and, further provides, air flow 236 from the vertical portion 210 that flows transverse relative to the air flow 236 from the horizontal portion 208, but not necessarily air flow that is precisely perpendicular relative to each other.

Further, the blower system 14 may provide air flow 236 in an alternating manner via an actuator 68 (FIG. 1) between the two chambers or first and second hollow spaces 224, 226 defined in the insert 204 so as to rock or jostle an infant on the insert 204 as well as provide substantially continuous air flow 236 to the infant regardless of the position of the infant on the insert 204. In addition, in the event an infant's face becomes positioned against a vertical portion 210 of the insert 204, the infant is provided additional air flow 236 through the apertures 202 defined in the inner liner 228 of the vertical portion 210 as well as the vertical portion 210 serving as a soft pad for the infant's head or body.

As in the previous embodiment (depicted in FIGS. 1 through 3), the blower system 14 and conduit 206 of this embodiment may include pressure and heart rate sensors as well as a temperature control to manage the air flow temperature for the infant. The conduit may also include, for example, two air flow channels (as depicted by broken line 238) for blowing air therethrough as well as an actuator for alternating air flow between the two air flow channels that funnel air into the first and second hollow spaces 224, 226, as well as any of the other structural and functional components described and depicted in FIGS. 1 through 3. Further, the insert 204, including the horizontal and vertical portions 208, 210, may be formed of a similar material as that described in the embodiment described in FIG. 1, or any other suitable materials as known to one of ordinary skill in the art.

In another embodiment, the conduit 206 may be optional such that the blower system 14 is directly coupled to the insert. The embodiment with an optional conduit may be implemented in any of the embodiments described herein, as known to one of ordinary skill in the art.

As depicted in FIG. 5C, another embodiment of a sleep surface insert system 201 is provided. This embodiment is substantially similar to that described and depicted in FIGS. 5 and 5A, except in this embodiment the insert 205 may include a single air-flow chamber 207 defined therein. The insert may include lateral connections 209 extending between the top liner 211 and the bottom liner 213 of the horizontal portion 215 as well as the inner liner 217 and the outer liner 219 of the vertical portion 221. Such lateral connections 209 may also extend between the liners to define channels of the single chamber 207. As in the previous embodiment, the inner liner 217 of the vertical portion 221 may define apertures 223 as well as the top liner 211 of the horizontal portion 215 may define apertures 223. In this embodiment, each of the apertures 223 communicate or are associated with the single chamber 207 to facilitate air flow from the blower system 14 (FIG. 1) such that apertures 223 in the horizontal portion 215 provide air flow upward in a general vertical direction, as depicted by arrows 225, and the apertures 223 defined in the vertical portion 221 of the insert may provide air flow in a general horizontal direction, as depicted by arrows 227.

The blower system 14 of this embodiment may be similar to that described and depicted in FIGS. 1 and 2, including the structural and functional components described therewith. Further, since the insert 205 of this embodiment has a single chamber, any conduit employed may only define a single channel therein and the sleep surface insert system 201 may not require an actuator, as in other embodiments described herein.

With respect to FIG. 6, another embodiment of the sleep surface insert system 200 is provided. In this embodiment, the insert 204 may be similar to that provided in the previous embodiment, but the system 200 may include a removable pad liner 250 for positioning and resting over the horizontal portion 208 and within the walls of the vertical portion 210 of the insert 204. The pad liner 250 may include a cushion portion 252 and an outer liner 254. The cushion portion 252 may be a foam material, such as an open cell foam or reticulated foam or similar material that facilitates air flow therethrough. The outer liner 254 may be a fabric or the like, such as a knitted or weaved material, that also readily facilitates air flow therethrough. The outer liner 254 may be coupled to an upper surface of the cushion portion 252 or be positioned around the cushion portion 252 in a sleeve-like manner. Such pad liner 250 may be removed from the insert 204 for purposes of cleaning such that the whole pad liner 250 may be washed or the outer liner 254 may be removed from the cushion portion 252 for cleaning the outer liner 254 separate from the cushion portion 252. With this arrangement, the pad liner 250 may be made of a material that provides additional comfort to an infant and also allows the flow of air from the insert 204 and through the pad liner 250 while also being removable for long term sanitary purposes.

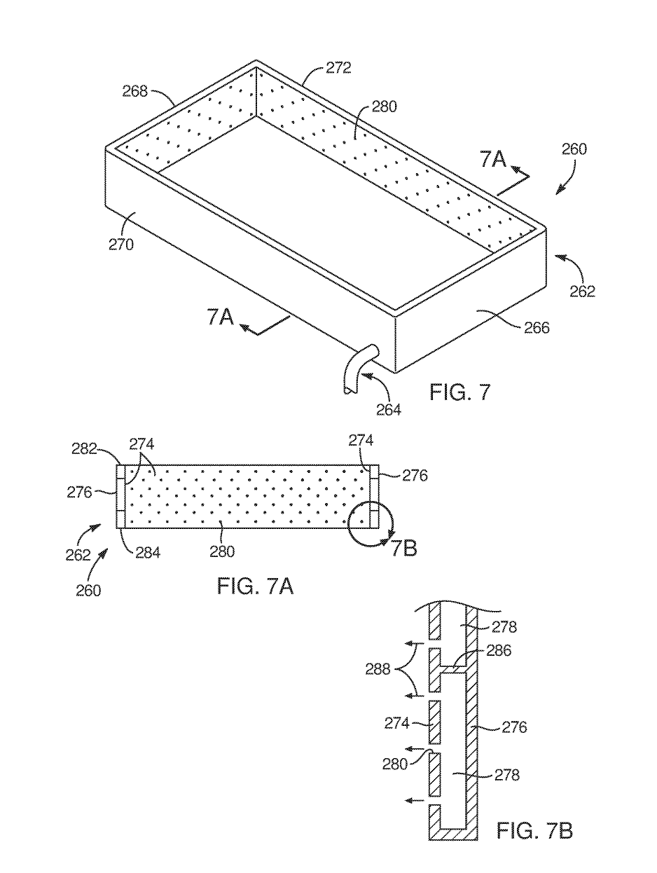

With reference to FIGS. 7, 7A, and 7B, another embodiment of an air flow system 260 is provided. The air flow system 260 is similar to the embodiment described and depicted in FIG. 5, except the air flow system 260 may not include air chambers defined and extending along a horizontal portion, but rather, includes air chambers defined and extending in a vertical portion 262 of the insert. The vertical portion 262 of the air flow system 260 of this embodiment may be sized and configured to act as a bumper pad in, for example, an infant's crib. The air flow system 260 may include the vertical portion 262, a blower system 14 (FIG. 1), and a conduit 264, the conduit 264 extending between the vertical portion 262 and the blower system 14. Such a blower system 14 and conduit 264 may include all or some of the structural and functional components described relative to the embodiment of FIG. 1.

The vertical portion 262 may include first and second facing vertical portions 266, 268 and third and fourth facing vertical portions 270, 272, each of which may border an interior side portion of an infant's crib (not shown). For example, each of the first, second, third and fourth vertical portions 266, 268, 270, 272 may be elongated to extend along one of the interior side portions of the crib as well as each extend with a vertical component to provide padding for an infant within the crib in the event, for example, the baby rolls over against one of the vertical portions. Each vertical portion may include an inner liner 274 and an outer liner 276 with one or more chambers or hollow spaces 278 defined therebetween.

The inner liner 274 may include perforations or apertures 280 and the outer liner 276 may be impermeable. Further, the inner liner 274 and outer liner 276 may be coupled together along a top end 282 and a bottom end 284 as well as include lateral connections 286 at various points between the top and bottom ends 282, 284 so that each vertical portion maintains a generally rectangular cross-sectional shape. As in previous embodiments, the inner liner 274 and the outer liner 276 may be formed of a polymeric material, or fabric, or a combination thereof. The lateral connections 286 and coupling along the top and bottom ends 282, 284 may be employed by thermal bonding or sewing or any other suitable means of coupling. With this arrangement, air flow 288 may be provided via the conduit 264 to the one or more hollow spaces 278 or chambers so as to provide air flow 288 from a vertically extending wall or inner liner 274 and through the apertures 280 such that the air flow 288 exits the vertically extending wall or inner liner 274 in a generally horizontal direction, as depicted with arrows in FIG. 7B. In this manner, the vertical portions 262 provide padding for an infant as well as provide air flow 288 to an infant if the infant's face moves against the vertical portion 262 of the air flow system 260.

The conduit 264 may include one or two flow paths depending on the number of chambers defined in the vertical portion of the air flow system 260. For example, in the case of a single chamber or hollow space defined in the vertical portion 262, the conduit 264 may include a single flow path through which air may be blown by the blower system 14 (FIG. 1). As in previous embodiments, in the case of two chambers or hollow spaces defined in the vertical portion 262, the conduit 264 may include two flow paths for funneling air to the two chambers and also may include the actuator to alternate inflation of the chambers.

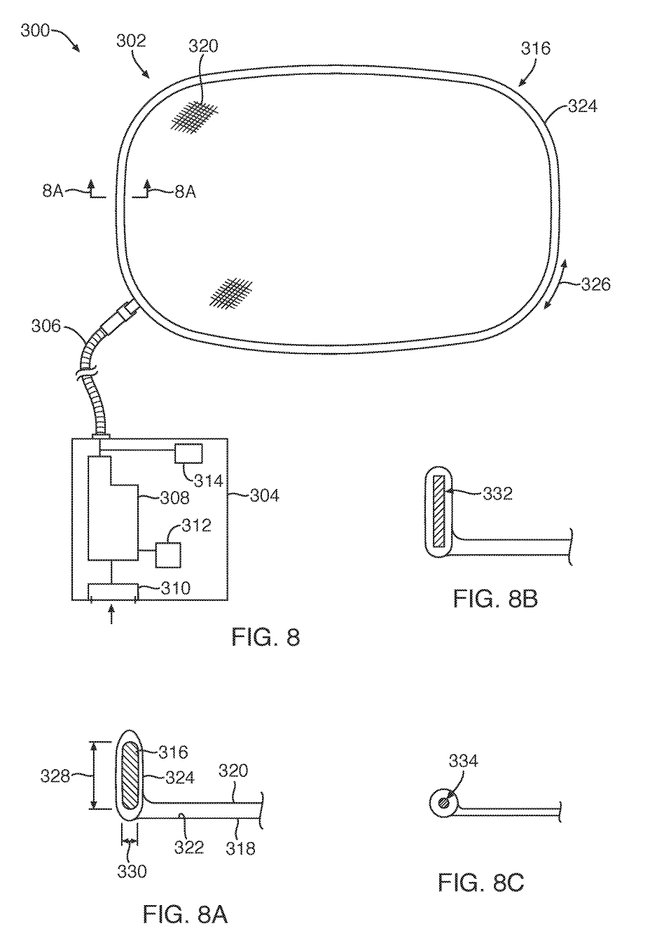

Now with reference to FIGS. 8 and 8A, another embodiment of a sleep surface insert system 300 is provided. The sleep surface insert system 300 may include an insert 302 and a forced air system 304 such that the forced air system 304 may be coupled to the insert 302 with a tube 306 or hose. The forced air system 304 may include a forced air device, such as a blower device 308, and various components that may include all or some of the components set forth for the blower system described relative to FIG. 1. For example, in one embodiment, the various components of the forced air system 304 may include a filter 310 and a manual controller 312 each coupled to or at least associated with the blower device 308. The tube 306 may be a kink-resistant hose or tube or any tube suitable for funneling air from the blower device 308 to the insert 302.

In another embodiment, the forced air system 304 may include a sensor 314 that may be associated with the insert 302. This sensor 314 may sense if an infant is positioned on or off the insert 302 such that the sensor 314 may automatically turn-off the blower device 308 upon the infant being removed. Similarly, upon positioning the infant on the insert 302, the sensor 314 may sense such positioning and automatically turn-on the blower device 308. The sensor 314 may be a pressure sensor, piezo electric sensor, and/or a temperature sensor to sense the presence the infant on the insert 302. The sensor may be made of sensitive materials, such as polyvinylidene difluoride (PVDF), or any other material suitable for acting as a sensor for sensing the presence of an infant or person on the insert 302 as known to one of ordinary skill in the art.

The insert 302 of the sleep surface insert system 300 may include a frame 316 that may be expandable so as to maintain the insert 302 in an expanded position. In the expanded position, the insert 302 may extend in a generally oval and flat configuration. In another embodiment, the insert 302 may extend in a generally rectangular and flat configuration. In still another embodiment, the insert 302 may extend in a generally rectangular and flat configuration with its corners rounded. As in previous embodiments, the insert 302 may include a bottom liner 318 and a top liner 320 to define a single hollow space 322 therebetween that communicates with the forced air system 304. In another embodiment, the insert 302 may define one or more hollow spaces or at least one hollow space, similar to previous embodiments herein, between the bottom liner 318 and the top liner 320.

The frame 316 of the insert 302 may extend in a continuous loop configuration along and adjacent a periphery of the top liner 320 and the bottom liner 318. The frame 316 may extend within a sleeve 324 coupled to the periphery of the top liner 320 and the bottom liner 318. The sleeve 324 may include a similar material as that provided for the bottom liner 318, the bottom liner 318 being non-permeable and the top liner 320 being a permeable membrane or a porous material, as in previous embodiments.

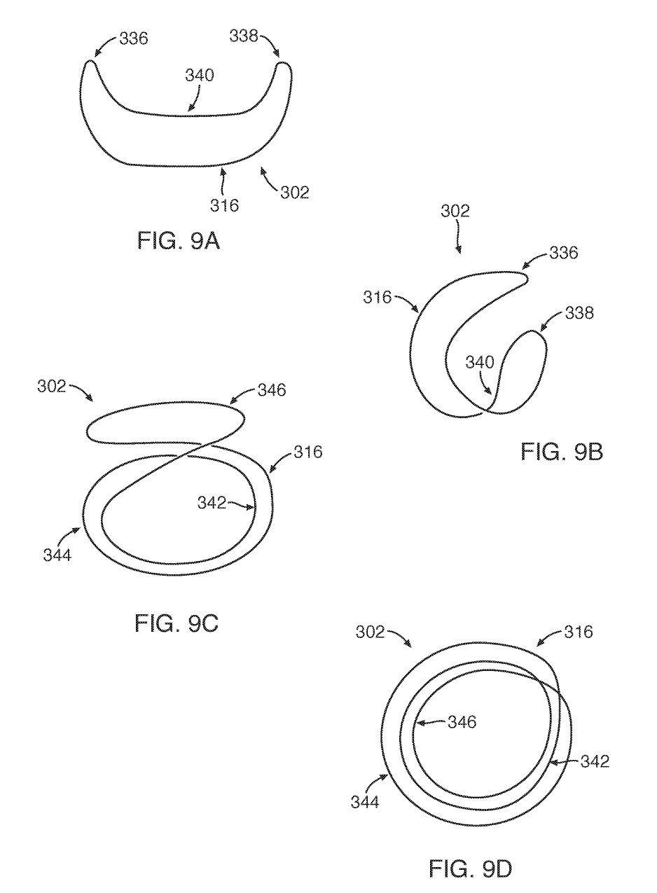

In one embodiment, the frame 316 may be a rigid material that may flex to various configurations. The frame 316 may be configured to move to an expanded configuration so as to maintain the top liner 320 in a spread-out and flat position. In another embodiment, the frame 316 may self-expand from a partially collapsed position to the expanded position. Further, the frame 316 may be moved between the expanded position or first position to a collapsed second position (see FIG. 9D). In the collapsed second position, the insert 302 may be readily stored and placed, for example, in a bag for storage. In another embodiment, the bottom liner 318 may include a sewn in pocket (not shown) sized to enclose or store the insert 302.

The frame 316 may include and extend with a radial length 326, a height 328, and a width 330. The radial length 326 may extend to exhibit the loop configuration. The width 330 may be smaller than the height 328 such that the frame 316 exhibits a flattened cross-section. Upon the frame 316 being in the expanded position, the height 328 may extend vertically and the width 330 may extend horizontally when the insert 302 is positioned horizontally over a sleep surface. In one embodiment, the frame 316 with the flattened cross-section may be an oval configuration. In another embodiment, a frame 332 may exhibit a flattened cross-section with a rectangular configuration, such as that depicted in FIG. 8B. In another embodiment, a frame 334 may exhibit a circular cross-section, as depicted in FIG. 8C, such that the frame exhibits a wire like frame.

With reference to FIG. 8A, as in previous embodiments, the top liner 320 may include a permeable membrane defining multiple pores (see, for example, FIG. 1A). The top liner 320 may be a material that facilitates air flow therethrough from the space defined between the top liner 320 and the bottom liner 318 of the insert 302. Such multiple pores may be miniature such that the multiple pores may only be viewable under magnification. The top liner may be formed, for example, from a bamboo fabric, nylon, polyester, cotton or any other commonly used bedding material, or any other suitable material that may facilitate air flow therethrough. The bottom liner 318 and the sleeve 324 carrying the frame 316 may be formed of a non-porous or non-permeable material to the extent that the bottom liner 318 does not readily facilitate air flow therethrough. For example, the bottom liner 318 may be formed from any suitable non-porous material, as known in the art. In another embodiment, the bottom liner 318 may also be formed of a porous material and may be the same material or similar material as the top liner 320, as set forth above. The frame 316 may be formed from a metal, or other substantially rigid material, such as a polymeric material, or the like, such as fiberglass. In another embodiment, the frame 316 may also include or be a foam material, or any other suitable material that wants to maintain a preferred position.

Now with reference to FIGS. 9A through 9D, a simplified version of the insert 302 with the frame 316 depicted in outline form is provided, illustrating a manner in which the frame 316 (and thus the insert 302) may be moved from the expanded first position (see FIG. 8) to the collapsed second position (FIG. 9D). As depicted in FIG. 9A, the frame 316 may be manually moved from the flat expanded position so that a first side end 336 and second side end 338 are folded upward toward a u-shape. As one continues toward moving the frame 316 to the u-shape, the rear side 340 will begin to collapse and the first side end 336 may more readily move upward, as shown in FIG. 9B. At this stage, the frame 316 may readily twist into multiple loops, such as three loops, such that the collapsing rear side 340 moves to an underside loop 342 which may be moved under a middle loop 344 with an upper loop 346 forming and suspended above the underside and middle loops 342, 344, as shown in FIG. 9C. The suspended upper loop 346 may then be pushed down over the middle and underside loops 344, 342, thereby, completely moving the frame 316 to a three-loop configuration or the collapsed second position, as shown in FIG. 9D. In another embodiment, the multiple loops of the collapsed second position may be a two-loop configuration. In another embodiment, the multiple loops of the collapsed second position may exhibit four-loops or more.

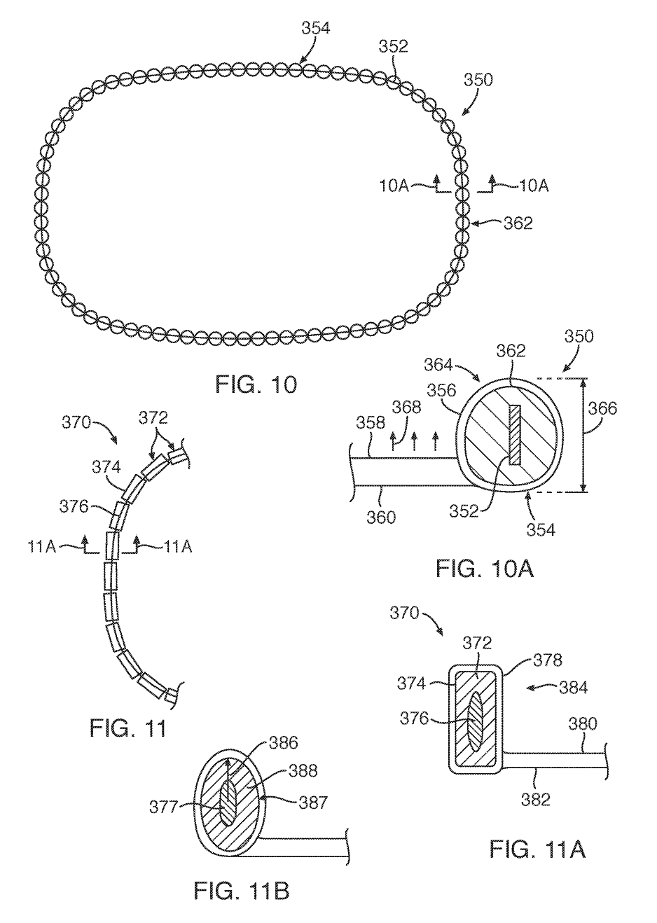

With reference to FIGS. 10 and 10A, another embodiment of an insert 350 for a sleep surface insert system is provided. The insert 350 of this embodiment may be employed with any sleep surface insert system described herein. This embodiment of the insert 350 with a frame 352 is similar to the insert of previous embodiments, except in this embodiment, the insert 350 may include a padding 354 over or surrounding the frame 352. FIG. 10 is a top view of the insert 350 (without the top and bottom liners and sleeve), only depicting the frame 352 and the padding 354 thereon for simplification purposes. FIG. 10A shows a cross-sectional view of FIG. 10 of the insert 350 with a sleeve 356, top liner 358 and bottom liner 360.

The insert 350 of this embodiment may include the frame 352 similar to that depicted in FIGS. 8A, 8B, and 8C, but in addition, includes the padding 354 extending along the frame 352. The padding 354 may be in the form of multiple spherical structures 362. The spherical structures 362 may be discrete, separate, and distinct spherical structures 362 with the frame 352 extending through a central portion of the spherical structures 362. Due to the spherical structures 362 being discrete relative to each other, the padding 354 may not inhibit the frame 352 such that the frame 352 may readily be moved from the expanded first position to the collapsed second position, as described relative to FIGS. 9A-9D. Similarly, with the padding 354 extending along the frame 352, the frame 352 may self-expand from a partially collapsed position to the expanded first position. Further, the spherical structures 362 may provide a more pronounced border 364 or rim with a height 366 that may be higher or above the top liner 358, similar to a bumper pad, for an infant sleeping on the insert 350 so as to provide padding 354 to the frame 352. In this manner, the padding 354 may provide the border 364 to assist and substantially ensure the infant remains on the insert 350 to receive air flow through the porous top liner 358, as indicated by arrows 368.

With reference to FIGS. 11 and 11A, another embodiment of an insert 370 similar to the previous embodiment is provided, except instead of spherical structures, a padding 372 may be in the form of elongated structures 374 with a rectangular or square cross-section. Similar to the previous embodiment, FIG. 11 is a partial top view of the insert 370 (without the top and bottom liners and sleeve), only depicting a frame 376 and the padding 372 thereon for simplification purposes. FIG. 11A shows a cross-sectional view of FIG. 11 of the insert 370 with a sleeve 378, top liner 380 and bottom liner 382 associated with the padding 372 and the frame 376.

Further, similar to the previous embodiment, each of the elongated structures 374 may be discrete, separate and distinct relative to each other so as to facilitate the frame 376 to move from the expanded first position to the collapsed second position. Further, the elongated structures 374 provide a bordered edge 384 or rim to the insert 370 or sleep surface that also provides padding 372 and assists maintaining the infant on the insert 370. In another embodiment, elongated structures 387 and padding 388 surrounding a frame 377 may include a radius 386 to exhibit a circular or oval cross-section, as depicted in FIG. 11B. It should be noted that any one of the embodiments of padding 354, 372, 388 set forth and described relative to FIGS. 10, 10A, 11, 11A, and 11B may include any one of the embodiments of the frame 316, 332, 334 as set forth and described relative to FIGS. 8A, 8B, and 8C.

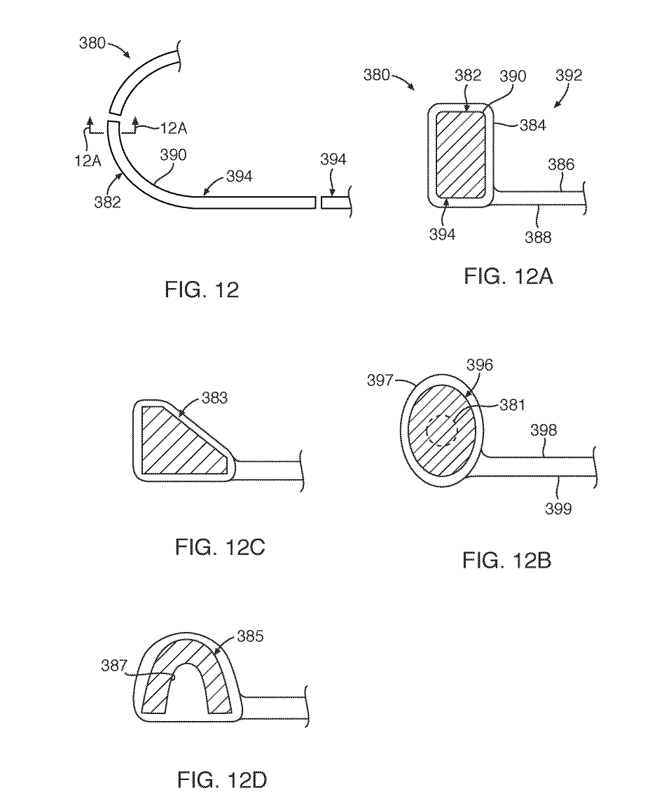

In another embodiment, as depicted in FIGS. 12 and 12A, an insert 380 may include padding structure 382 sized to be positioned within a sleeve 384 and adjacent a top liner 386 and bottom liner 388, FIG. 12 not including the sleeve and top and bottom liners, for simplification purposes. The padding structure 382 may be in the form of elongated structures 390, discrete relative to each other, and may act as a bordered structure 392 to the sleep surface or top liner 386 of the insert 380. This padding structure 382 may act as a frame 394 to the insert 380 to maintain the insert 380 in the expanded first position. Further, due to the elongated structures 390 being discrete, the insert 380 may be manually folded to a collapsed position. In this embodiment, the insert 380 may be manually unfolded from the collapsed position and manually spread-out and positioned in the expanded first position. Further, the padding structure 382, as a whole, may be stiffer, stronger, and/or heavier than the bottom liner 388 and the top liner 386 associated therewith such that the padding structure 382 may maintain the insert 380 in the expanded position. As depicted in FIG. 12A, the discrete elongated structures 390 may include a rectangular cross-section and positioned within the sleeve 384.

In another embodiment, discrete elongated structures 396 or frame may include a circular or oval cross-section and positioned within a sleeve 397 and adjacently positioned top and bottom liners 398, 399, as depicted in FIG. 12B. As depicted in FIGS. 12A and 12B, the discrete elongated structures 390, 396 may be solid or a continuous structure along the lateral cross-section of the elongated structures. In another embodiment, the elongated structures may include a hollow region 381 extending longitudinally along an axis of the elongated structures, as indicated by dotted line in FIG. 12B. The hollow region 381 may act as a lumen to facilitate air flow therethrough.

With respect to FIG. 12C, in another embodiment, discrete elongated structures, acting as a frame, may include a wedge shaped lateral cross-section. In another embodiment, discrete elongated structures may define a c-shape or u-shaped lateral cross-section, the elongated structures being the frame for the insert, as depicted in FIG. 12D. The c-shape or u-shaped cross-section may define a lumen or hollow region that may be utilized for air flow through the frame. Such hollow region, similar to the hollow region 381 depicted in FIG. 12B, may also be employed with the embodiments depicted in FIGS. 12A and 12C.

With respect to the padding 354, 372, 382 depicted in FIGS. 10, 11, and 12, the padding may be a foam material, such as polyurethane, polyester, or silicon foam, or any other suitable padding or soft material that will substantially maintain its shape. Further, the various insert embodiments set forth herein may be employed with any of the embodiments of a blower system or forced air system described herein, such as the forced air system described relative to FIG. 8.

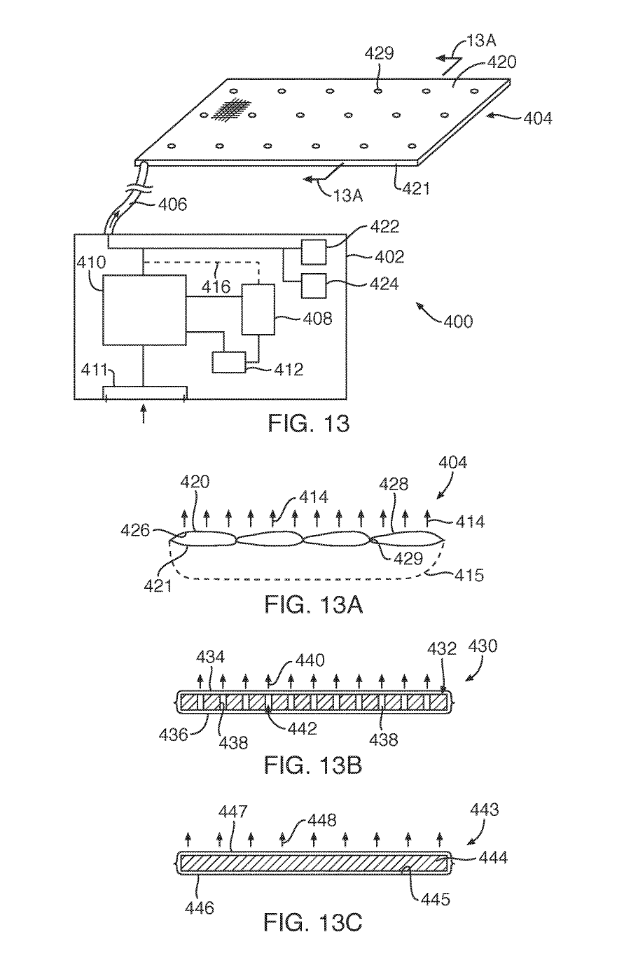

Now with reference to FIGS. 13 and 13A, another embodiment of a sleep surface insert system 400 is provided. The sleep surface insert system 400 of this embodiment may include a forced air system 402 and an insert 404 with tubing 406 extending between the forced air system 402 and the insert 404. The forced air system 402 may also include a filter 411, similar to previous embodiments. In this embodiment, the forced air system 402 may be coupled to or associated with a gas source 408, such as a tank or any suitable pressurized gas source or chamber that may hold, for example, oxygen or nitrogen, or any other gas, such as argon, or combination of gases. In one embodiment, the pressurized source may be a gas source that may be delivered through a gas port in a wall at a hospital, for example. In addition, the forced air system 402 of this embodiment may include all or some of the components set forth relative to the other forced air or blower systems described herein. With this arrangement, the forced air system 402 may be employed to provide an oxygen rich atmosphere above a top liner 420 of the insert 404 via the oxygen flowing from the gas source 408. Such oxygen rich atmosphere may be useful for patients in a hospital, for example. Similarly, the forced air system 402 may be employed to provide an oxygen poor atmosphere above the top liner 420 via the nitrogen in the gas source 408, or any other suitable inert gas, such as argon or combination of gases, that may flow from the gas source 408. Such oxygen poor atmosphere may be employed for athletic training purposes, e.g., altitude training, or the like, to encourage increased red blood cell formation and hematocrit increase.

The forced air system 402 may include a forced air device or blower device 410 with the gas source 408 associated therewith. The forced air system 402 may also include a manual control 412 that may be associated with the blower device 410 and/or gas source 408. In one embodiment, as the blower device 410 is flowing air from the intake to the tubing 406 and then through pores of the top liner 420 of the insert 404, the gas source 408 may feed its contents into the air stream of the blower device 410 to push the oxygen or nitrogen to the insert 404 and out the pores, as indicated by arrows 414.

In another embodiment, as depicted by dotted line 416, the gas source 408 may directly feed the insert 404 via the tubing 406 such that the pressurized state of the gas source 408 may act to force gas through the tubing 406 to the insert 404 and through the pores defined in the top liner 420 of the insert 404. In this embodiment, it may not be necessary to include the blower device 410 with the forced air system 402 since the pressurized state of the gas source 408 is forcing the gas through the pores. In other words, the gas source 408 may be independent and/or exclusive of a blower device. In either embodiment, the forced air system 402 may include a sensor 422 and a display 424. The sensor 422 may be coupled downstream of the blower device 410 and/or the gas source 408. Further, the sensor 422 may be configured to sense the gas content and levels being dispensed to the insert 404. The display 424 may provide information relative to the sensed data so that a user may manually modify the content and levels appropriately.

The insert 404 of the sleep surface insert system 400 of this embodiment may be dimensioned and sized similar to a surface area of a typical pillow or larger. For example, the insert 404 may be positioned directly over a pillow for a user to lay his/her head thereon. In one embodiment, the insert 404 can function as a pillow with the appropriate padding (see FIGS. 13B and 13C). In another embodiment, the insert 404 may be sized to be inserted within a pillow case to be positioned alongside a pillow. In another embodiment, the insert 404 may include a sleeve 415 (as depicted with dotted line) alongside, for example, the bottom liner 421 that may be sized to receive a padded pillow. Upon the forced air system 402 being turned on, the pores defined in the top liner 420 of the insert 404 may facilitate air flow to, thereby, provide an oxygen rich or oxygen poor atmosphere above and adjacent the insert for the user to inhale the air therefrom.

In one embodiment, the insert 404 may include the top liner 420 and a bottom liner 421 with an air space 426 therebetween, similar to previous embodiments. Further, the top liner 420 may include pores defined therein extending between an exterior surface 428 of the top liner 420 and the air space 426 between the bottom liner 421 and the top liner 420. The top liner 420 may be coupled to the bottom liner 421 at a periphery of each of the top and bottom liners 420, 421. Further, the top liner 420 and the bottom liner 421 may be coupled at various point attachments 430, or line attachments, within the periphery of the top and bottom liners 420, 421. With the point attachments 430, the insert 404 may maintain a generally flat shape as air flows into the space 426 and through the pores. Such point attachments 430 may be employed in other embodiments set forth herein.

In another embodiment, as depicted in FIG. 13B, an insert 430 may include a padding 432 positioned between a top liner 434 and a bottom liner 436. The padding 432 may include channels 438 defined therein along which air may flow from the forced air system 402 (FIG. 13) to then disperse through pores defined in the top liner 434, as indicated by arrows 440. For example, the channels 438 may extend along a length of the insert 430 with at least one channel extending along a width of the insert 430. In this manner, the channels 438 may each be interconnected to define a single hollow space 442 within the insert 430 such that the tubing 406 (FIG. 13) directly feeds the single hollow space 442. The padding 432 may be a foam material, such as polyurethane foam. The foam material may be a reticulated foam or non-reticulated foam. In the case of the padding 432 being non-reticulated foam, the air may move along the channels 438. In the case of the padding 432 being reticulated foam, the air flow may move along the channels 438 as well as through the reticulated foam or padding 432. The padding 432 may include a height to act as padding 432 for a pillow for comfort purposes as well as for purposes of maintaining a shape of the insert 430.

With respect to FIG. 13C, in another embodiment, an insert 443 may include a padding 444 that may not include the formed and defined channels, as in the previous embodiment. For example, the padding 444 may extend to substantially fill a space 445 defined between a bottom liner 446 and a top liner 447, the top liner 447 including pores. In one embodiment, the padding 444 may extend continuously in the space 445 so as to define a single pad. The padding 444 in this embodiment may be reticulated foam, such as reticulated polyurethane foam, so as to facilitate air flow through the padding 444 and then through the pores defined in the top liner 447, as indicated by arrows 448. In another embodiment, the padding 444 may include multiple layers of padding 444. In a version of multiple layers of padding 444, the padding 444 disposed adjacent the top liner 447 may be reticulated foam and the padding 444 positioned adjacent the bottom liner 446 may be, for example, reticulated foam or non-reticulated foam or the like.

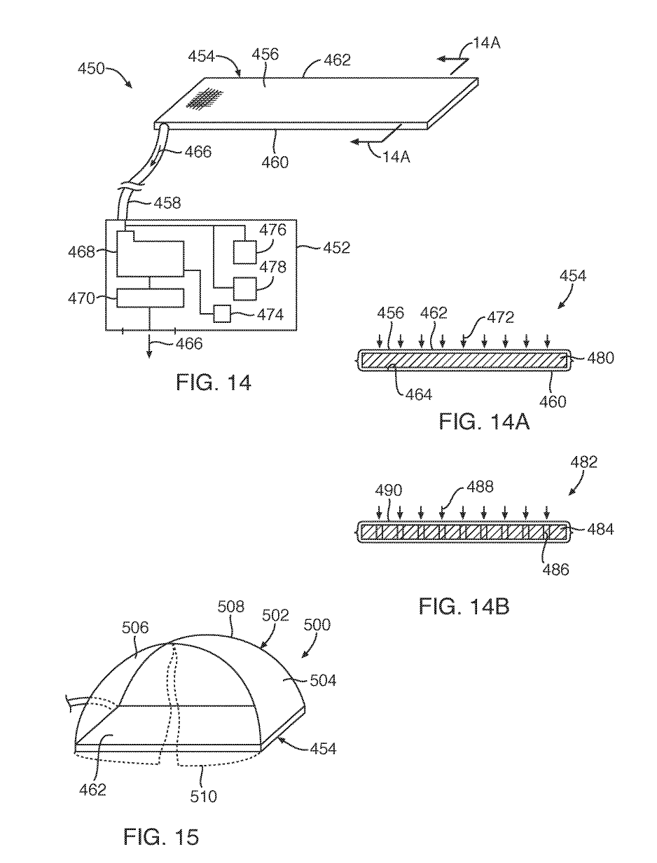

With reference to FIGS. 14 and 14A, another embodiment of a sleep surface insert system 450 is provided. This embodiment may be similar to previous embodiments, except a forced air system 452 may suction air from an insert 454, rather than blow air toward the insert. Such suctioning of air may be employed for suctioning unwanted air and/or agent away from an infant or patient, such as carbon dioxide or anesthetic or any other agent desired to be removed that may be exhaled from a patient, for example. In this manner, an infant or patient's head may be positioned adjacent an exterior surface 456 of the insert 454 such that air exhaled from the infant or patient may be suctioned therefrom. The sleep surface insert system 450 of this embodiment may include the forced air system 452 and the insert 454 with a tube 458 therebetween.

As in previous embodiments, the insert 454 may include a bottom liner 460 and a top liner 462 that may define a space 464 therebetween, the top liner 462 including pores defined therein. The tube 458 may be coupled to the insert 454 and coupled to the forced air system 452 to facilitate air flow from the insert 454 to the forced air system 452, as indicated by arrow 466. The tube 458 or hose may be a kink-resistant tube, as known in the art, and may include coupling joints at ends of the tube 458 that facilitate quick connection and disconnection of the tube 458 from the insert 454 and/or the forced air system 452.

The forced air system 452 may include a forced air device or suction device 468 and a filter 470. The suction device 468 may be sized and configured to provide suction necessary to pull air and an agent through the pores defined in the top liner 462, as indicated by arrows 472. The filter 470 may be an absorbent filter, such as a charcoal filter, or molecular sieve/filter or any suitable absorbent filter known in the art. The filter 470 may be sized and configured to absorb an agent, such as carbon dioxide from an infant or anesthetic from a patient, that may be suctioned by the suction device 468. As depicted, the filter 470 may be positioned downstream of the suction device 468. In another embodiment, the filter 470 may be upstream of the suction device 468 within the forced air system 452. In still another embodiment, the filter 470 may be positioned along the tube 458 or adjacent ends of the tube 458. In another embodiment, the suction device 468 may simply remove the suctioned air from the insert 454 and completely from the room with, for example, tubing.

The forced air system 452 may include a manual control 474 sized and configured to control the suction device 468, for example. The forced air system 452 may also include a sensor 476 and a display 478. The sensor 476 may be configured to sense the agent being pulled or suctioned from the insert 454. In one embodiment, the sensor 476 may be positioned upstream of the suction device 468, as depicted, and with the forced air system 452. In another embodiment, the sensor 476 may be positioned adjacent to and/or upstream of the filter 470 and within the forced air system 452. In still another embodiment, the sensor 476 may be positioned along the tube 458 or adjacent the couplings at ends of the tube 458. The display 478 may provide data relative to the sensed agent. For example, upon a patient completing surgery, the patient may be positioned on the insert 454 and may be exhaling an agent, such as anesthetic. The suction device 468 may pull the exhaled agent through the pores and from the insert 454 such that the agent may be carried to the filter 470, during which the display 478 may be monitored to determine when the agent has been substantially or fully expelled from the patient. In this manner, the sensor 476 and display 478 associated with the forced air system 452 may provide information regarding a patient. Similarly, the sleep surface insert system 450 may be employed with an infant such that the system may pull air from above the insert 454, such as exhaled carbon dioxide from the infant, while also monitoring the infant with the sensor 476, in which the sleep surface insert system 450 may include any one of the sensors set forth and described relative to any one of the embodiments herein.

With respect to FIG. 14A, the insert 454, as in the previous embodiment, may include a padding 480 positioned between the bottom liner 460 and the top liner 462. The padding 480 may be sized and configured to facilitate air flow therethrough such that an agent and air may be pulled through the pores, through the padding 480, and toward the suction device 468 (FIG. 14). The padding 480 may be a single pad or multiple adjacently positioned pads disposed side-by-side or layered. In one embodiment, the padding 480 may be a foam material. The foam material may include a reticulated foam, such as reticulated polyurethane foam, sized and configured to facilitate air flow, as indicated by arrows 472, through the reticulated foam.

In another embodiment, as depicted in FIG. 14B, an insert 482 may include a padding 484 that may be sized and arranged to define channels 486 within the padding 484 and insert 482, similar to that set forth and described relative to FIG. 13B. The channels 486 defined in the padding 484 or in the insert 482 may be sized and configured to facilitate air flow, as indicated by arrows 488, from above a top liner 490, through the pores, then through the channels 486, and toward the suction device 468 (FIG. 14). As in previous embodiments, the padding 484 may be sized and configured to facilitate air flow therethrough or may be substantially impervious to air flow. For example, the padding 484 may be a foam material. In one embodiment, the padding may be a reticulated foam. In another embodiment, the padding may be a non-reticulated foam. The foam material may be a polyurethane foam or any other suitable foam material or the like.

As in previous embodiments, the insert 454, 482 depicted in FIGS. 14A and 14B can function as a pillow or be associated with a pillow. As previously set forth, in one embodiment, the insert 454, 482 can function as a pillow with the appropriate padding. In another embodiment, the insert 454, 482 may be sized to be inserted within a pillow case to be positioned alongside a pillow. In another embodiment, the insert 404 may include a sleeve (similar to that set forth in FIG. 13A) alongside, for example, the bottom liner 421 that may be sized to receive a padded pillow.

With respect to FIG. 15, in another embodiment, the sleep surface insert system 450, as described and depicted in FIG. 14, may include a shroud 500 that may be extend above the insert 454. For example, the shroud 500 may include a shroud frame member 502 and a shroud member 504. The shroud frame member 502 may, for example, include a first frame member 506 and a second frame member 508, each extending from the corners of the insert 454. The first frame member 506 may extend from front corners of the insert 454 and the second frame member 508 may extend from the rear corners of the insert 454, each extending in a bowing manner. The shroud member 504 or shroud material may extend over the insert 454 and from the first frame member 506 and the second frame member 508. Further, the shroud member 504 may extend vertically from the second frame member 508 and above the insert 454. The shroud member 504 may also extend to define a somewhat loose curtain 510 that may extend loosely from the first frame member 506 above a front side of the insert 454. With the shroud 500 disposed above the insert 454, a person's head, such as a patient, may be positioned over the insert 454 and surrounded by the shroud 500 so that the forced air system 452 may suction, through the pores defined in the top liner 462 of the insert 454, substantially that which is exhaled from the person, the shroud 500 substantially maintaining the exhaled air from escaping into the atmosphere of the room exterior to the shroud 500 and the sleep surface insert system 450 (see also, FIG. 14). This embodiment of the insert 454 may be sized similar to the dimensions of a pillow or larger. Further, the shroud 500 of this embodiment may be employed with the sleep surface insert system described and depicted relative to FIGS. 13, 13A, 13B, and 13C, or any other of the embodiments described herein. The sleep surface insert system, as set forth in any one of the various embodiments herein, is readily removable from the sleep surface that the insert may be positioned upon or over, the sleep surface insert system being sizeable for any one of a pillow, a bed, or an infant's crib, such that the sleep surface insert system is portable.

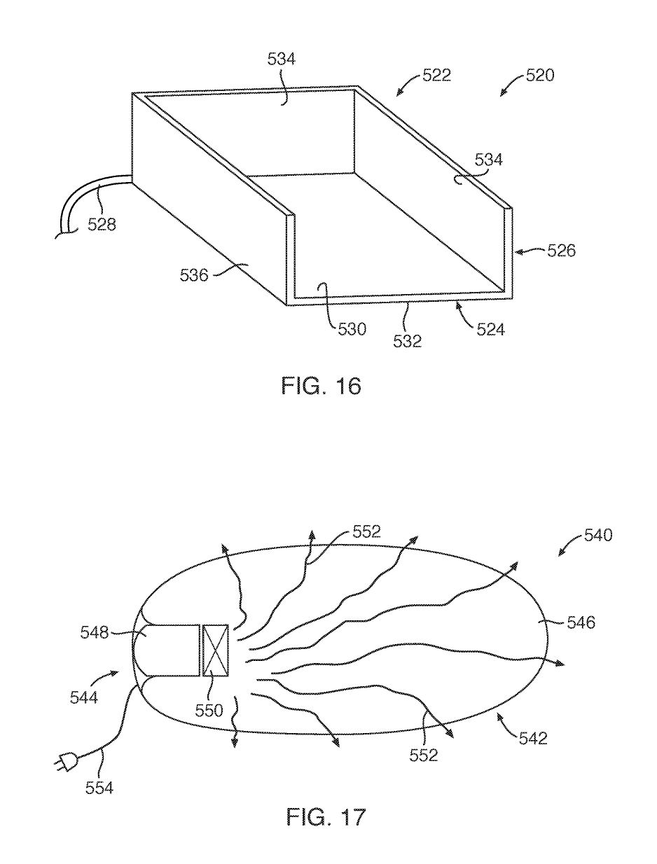

With reference to FIG. 16, in another embodiment, an insert 522 of a sleep surface insert system 520 is provided. The insert 522 of this embodiment may include a single space or one or more spaces within the insert, as in previous embodiments. In this embodiment, the insert 522 may include a floor 524 and three side walls 526. Each of the floor 524 and three side walls 526 may act as the insert 522 to flow air therethrough. The insert 522 may flow air via a tube 528 and a forced air system (not shown), similar to that depicted in previous embodiments. The floor 524 may include a top liner 530 and a bottom liner 532 and each of the side walls 526 may include inner liners 534 and outer liners 536. The top liner 530 and the inner liners 534 may be porous to facilitate air flow, similar to that described in previous embodiments. The insert 522 of this embodiment may be sized and configured to receive, for example, a person's head. Further, the insert 522 may be employed with a forced air system (not shown) that facilitates both pressure and suction, such as the forced air systems 402, 452 described and depicted in FIGS. 13 and 14. With this arrangement, the sleep surface insert system 520 of this embodiment may be employed for similar purposes described relative to FIGS. 13 and 14, or for other similar purposes as described herein.

Now with reference to FIG. 17, in another embodiment, an insert 542 of a sleep surface insert system 540 is provided. The insert 542 of this embodiment may include the forced air system 544 integrated within the insert 542, essentially eliminating the tube of previous embodiments and internalizing the forced air system 544. In one embodiment, the insert 542 includes structure similar to a pillow such that the insert may include a padding structure 546, such as foam or the like. The forced air system 544 may include a forced air device 548 and may optionally also include a filter 550, similar to previous embodiments, except the forced air device 548 and filter 550 may be housed within the insert 542. The forced air device 548 may be a blower device, a suction device, or a pressurized tank, for example. The filter 550 may be an absorbent filter, such as an absorbent charcoal filter or molecular sieve. The air flow may pass through the filter 550 and through padding structure 546, which may include reticulated foam and/or lumens defined in the pillow for air to diffuse therethrough, as indicated by arrows 552. Alternatively, in the case of a suction device, air may be suctioned from the external surface of the insert 542 and flow to the forced air device 548. The sleep surface insert system 540 may include means to be powered, such as with a plug 554, or the sleep surface insert system 540 may be battery powered, for example. In another embodiment, the forced air system 544 may be internalized with an insert along the frame or within an upstanding side wall along the frame, making the sleep surface insert system for an infant much more compact and portable.

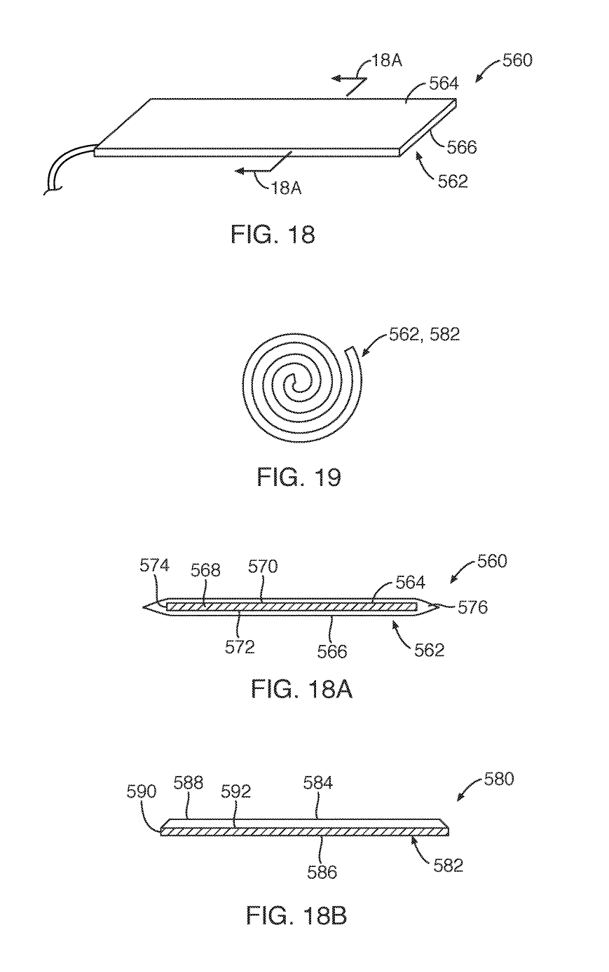

Now with reference to FIGS. 18 and 18A, another embodiment of an insert 562 of a sleep surface insert system 560 is provided. The insert 562 of this embodiment may be coupled with a forced air system (not shown), which may include all or some of the components of the forced air system embodiments disclosed herein. In this embodiment, the insert 562 may include a top liner 564 and a bottom liner 566 with a padded member 568 therebetween. The insert 562 may define a single space 576 or multiple spaces defined between the top liner 564 and the bottom liner 566. As in previous embodiments, the top liner 564 may include multiple pores and be made from a porous material. In one embodiment, the bottom liner 566 may also include multiple pores and be made from a porous material, similar to the top liner 564. In this manner, the top liner 564 or the bottom liner 566 may act as an upward facing surface to ensure an infant is laying on an effective surface providing air flow through the upward facing surface of the insert 562. In another embodiment, the bottom liner 566 may be non-porous.

The padded member 568 may be disposed between the top liner 564 and the bottom liner 566 such that a top side 570 and bottom side 572 of the padded member 568 may extend generally parallel and planar relative to the top liner 564 and the bottom liner 566. The padded member 568 may include a periphery 574 sized smaller than the periphery of the top liner 564 and the bottom liner 566. The periphery 574 of the padded member 568 may be sized such that the air space 576 may extend around the periphery 574 between the top and bottom sides 570, 572 of the padded member 568. The padded member 568 may include point or line attachments between the top side 570 of the padded member 568 and the top liner 564. Similarly, there may be point or line attachments between the bottom side 572 of the padded member 568 and the bottom liner 566. The padded member 568 may be flexible so as to be moveable between an expanded position and a compact position. In the expanded position, the padded member 568 is flat and substantially planar such that the padded member 568 may be configured to maintain its shape or prefers to maintain its substantially flat and planar configuration. The padded member 568 may be sized and configured to assist the insert in maintaining a spread-out and flat position. The padded member 568 may be formed of a polymeric material or any other suitable flexible, soft material, such as an elastomer material made from a thermoplastic elastomer or a foam material made from, for example, polyurethane.

With respect to FIG. 18B, another embodiment, of an insert 582 of a sleep surface insert system 580, similar to the previous embodiment. In this embodiment, the insert 582 may include a top liner 584 and a bottom liner 586, the top liner 584 defining multiple pores such that the top liner 584 may include a permeable or porous material. Further, as in previous embodiments, the top liner 584 and the bottom liner 586 define a space 588 therebetween. The bottom liner 586 may be in the form of a padded member, similar in all respect to that described for the padded member of the previous embodiment. The periphery of the top liner 584 may be coupled to a periphery 590 of the bottom liner 586 via sewing, heat-setting, or any other suitable coupling process as known in the art. The top liner 584 may include multiple point or line attachments to a top side 592 of the bottom liner 586. As set forth, the bottom liner 586 may be in the form of a padded member, as previously described, and may be a heavier material than the top liner 584 such that the bottom liner 586 may be configured to maintain the top liner 584 in a spread-out and flat position, or use position. Further, the insert 582 may be moved to a compact position after, for example, decoupling the forced air system.

In one embodiment, the insert 562, 582 of FIGS. 18A and 18B, may be rolled-up from one end to move the insert 562, 582 to the compact position, as depicted in FIG. 19. In another embodiment, the padded member of the insert 562, 582 described in FIGS. 18A and 18B may be segmented to define discrete padded portions or include thinned portions such that the padded member of the insert 562, 582 includes padded portions segmented in a manner to facilitate the insert to be folded to be positioned into the compact position.

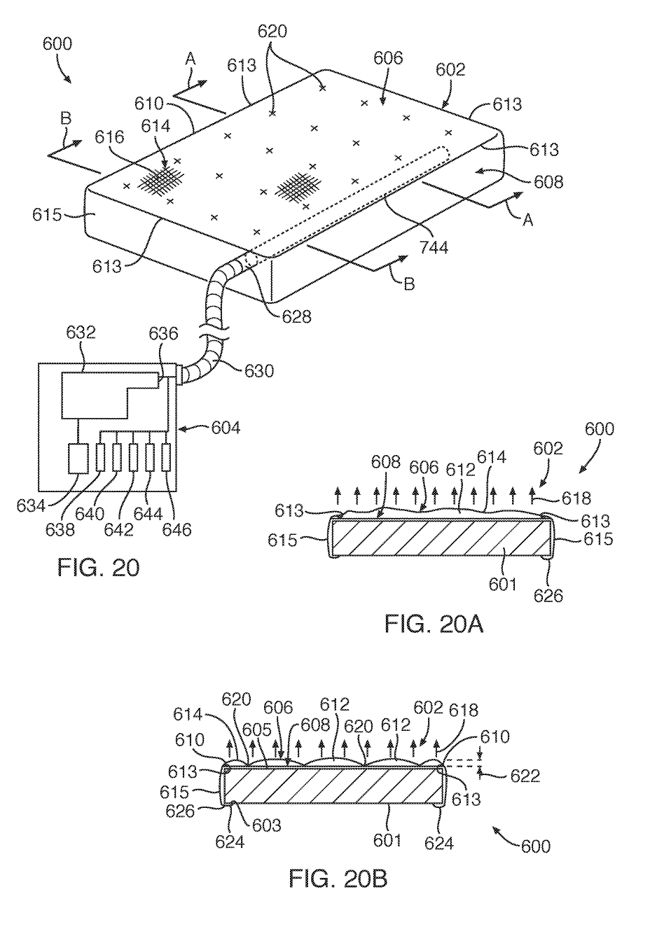

Now with reference to FIGS. 20, 20A and 20B, another embodiment of a sleep surface system 600 is provided. In this embodiment, the sleep surface system 600 may be sized to be positioned and attached to an infant's mattress 601 or sleep surface. In one embodiment, the sleep surface system 600 may include an insert 602 and a forced air device 604 coupled to the insert 602 at a port opening 628, the forced air device 604 forcing air to and through the insert 602. As in previous embodiments, the insert 602 may be removable so as to be portable and readily washable such that the insert 602 may be readily removed from the forced air device 604 and removed from the infant's mattress 601 or sleep surface. Further, the insert 602 of this embodiment may be sized and configured with structure to act similar to a fitted sheet.

The insert 602 may include a top liner 606 and a bottom liner 608 such that a top liner periphery 610 may be coupled or directly attached to the bottom liner 608 to define a substantially enclosed space 612 between the top liner 606 and the bottom liner 608. In other words, the enclosed space 612 may be defined by the top liner 606 and the bottom liner 608 with a peripheral boundary 613 or peripheral junction defined by the coupling between the top and bottom liners 606, 608. Such peripheral boundary 613 may be, for example, a continuous coupling (or substantially continuous coupling due to the port opening 628 that may be positioned along the peripheral boundary), such as a threaded stitching or bonding between the top and bottom liners, that may extend from the port opening 628 (or adjacent thereto) of the insert 602 to generally extend and define a rectangular area sized similar to the top surface of an infant's mattress.

The top liner 606 may be formed of a woven material 614 such that a weave of the material may have a density or tightness to the woven material so as to inherently define pores 616 therein within the material itself. Such woven material 614 may be a natural material or fiber, such as cotton or bamboo, or such woven material 614 may be formed of a synthetic polymeric material or synthetic blend between polymeric and natural fibers. The pores 616 defined in the woven material 614 may be sized so as to facilitate air flow therethrough, as depicted by arrows 618, from the space 612 defined in the insert 602.

The top liner 606 may also be attached to the bottom liner 608 at multiple separate and discrete attachment locations 620 that may be positioned separate from the peripheral boundary 613 between the top and bottom liners 606, 608. Such multiple discrete attachment locations 620 of the top liner 606 to the bottom liner 608 may exhibit an array of discrete attachment locations 620 and may be direct attachments, for example, with a stitch of one or more threads in a knotting manner. Further, the multiple separate and discrete attachment locations 620 may be point or line attachments or the like. With the array of attachment locations 620, separate from the peripheral boundary, the top liner 606 may be maintained at a height 622 relative to the bottom liner 608 as air flows within the space 612 between the top liner 606 and the bottom liner 608.

As previously set forth, the insert 602 of this embodiment may be sized to exhibit structure similar to a fitted sheet. In this manner, the bottom liner 608 may include a bottom liner periphery 624 that may be larger than the top liner periphery 610 such that the bottom liner 608 may be sized to extend around end portions of an infant's mattress 601. For example, the bottom liner periphery 624 may include an elastic portion 626 along portions of the bottom liner periphery 624. Such portions with the elastic portions 626 attached thereto may be sized and configured to attach to an underside 603, such as underside corners and periphery, of an infant's mattress 601. The top liner periphery 610 of the top liner 606 may be sized to extend smaller than the bottom liner periphery 624 such that the top liner 606 may be sized and positioned substantially along a top surface 605 of the infant's mattress 601. In this manner, the top liner 606 may be configured to be positioned substantially horizontally over the infant's mattress 601 and the bottom liner 608 may exhibit features similar to a fitted sheet so as to extend over the top surface 605, around vertical sides and to an underside 603 of the infant's mattress 601.

In another embodiment, the top liner 606 may extend with a larger periphery than the bottom liner 608 such that the top liner 606 extends with the fitted sheet structure so as to extend over the top surface 605 of the infant's mattress 601, around the vertical sides, and to an underside 603 of the infant's mattress 601, the top liner 606 having the elastic portions 626 along portions of the periphery of the top liner 606. In this embodiment, the bottom liner 608 may be sized similar to the size of the top surface 605 of the infant's mattress 601. Alternatively, in another embodiment, the top and bottom liners 606, 608 may be similarly sized (and may substantially correspond with the size or periphery of the top surface 605 of the infant's mattress 601), coupled to each other at their respective peripheries with a supplementary liner portion extending from the respective peripheries of the top and bottom liners 606, 608. Such supplementary liner portion may be sized and configured to be tucked around the vertical sides and to the underside 603 of the infant's mattress 601 and may include the elastic portions 626 attached thereto. The supplementary liner portion extending from the top and bottom liners 606, 608 may exhibit structure such that the insert extends similar to a fitted sheet. In each of these embodiments of the sleep surface system 600, the insert may exhibit a supplementary liner portion or peripheral portion 615 extending from the peripheral boundary 613 between the top and bottom liners 606, 608 as an additional material portion to extend over the vertical sides to an underside 603 of the infant's mattress 601 similar to a fitted sheet. In another embodiment, the insert 602 with its top liner 606 and bottom liner 608 defining a space 612 therebetween may be sized so that the periphery of the insert 602 generally corresponds with the size of the top surface 605 of an infant's mattress 601 without the before-described peripheral portion 615 extending from the peripheral boundary 613 of the insert 602. Rather, the insert 602 may include an elastic portion extending from adjacent corners of the insert to facilitate temporarily attaching the insert 602 to the infant's mattress 601.

In one embodiment, the bottom liner 608 may be non-porous or formed of an impermeable material, meaning the bottom liner 608 may be configured to substantially prevent air flow therethrough. Such bottom liner 608 may also be formed of a material to substantially prevent moisture through the material of the bottom liner 608. The bottom liner 608 may be formed from a polymer, such as, polyester or nylon or the bottom liner may be formed of a material with a coating to substantially prevent air flow through any pores that may be inherently within the bottom liner 608. In another embodiment, the bottom liner 608 may be formed with a similar material as the top liner so as to include pores therein.

The insert 602, as in previous embodiments, may include a port opening 628 sized and configured to channel air flow from the forced air device 604 to the space 612 between the top and bottom liners 606, 608. The port opening 628 may be sized and configured to couple directly to the forced air device 604 or may be coupled to a hose 630 positioned between the forced air device 604 and the port opening 628. The port opening 604 may be positioned at any location along the top liner periphery 610 to provide an opening between the top liner 606 and bottom liner 608 to force air within the space 612 defined in the insert 602.

In one embodiment, the forced air device 604 may include a blower fan 632 and manual controls 634 with a fan output 636 coupled directly to the port opening 628 or to the hose 630. Such manual controls 634 may include features such as fan speed and/or a power switch for turning the blower fan 632 on and off. In another embodiment, the forced air device 604 may also be associated with a filter 638, such as a Hepa filter or the like. Such filter 638 may be positioned over the path of the air flow generated by the blower fan 632, such as being directly coupled to the blower fan 632, the hose 630, or a plenum 744 (shown in dashed lines), the plenum discussed in further detail below. In another embodiment, the forced air device 604 may include a temperature sensor 640, one or more controllers 642, and a heating element 644, similar to that set forth in previous embodiments herein, that may facilitate a user to control temperature of the air moving through the insert 602. Further, other sensors 646 (e.g., pressure sensor, heart rate sensor, etc.) may be employed with the sleep surface system 600, similar to that described in other embodiments herein. With this arrangement, the pores 616 defined in the woven material 614 may be sized and configured to allow air flow therethrough at a rate suitable to move the air adjacent to an infant's face to, thereby, limit and potential carbon dioxide pockets that may form adjacent to an infant's face while also controlling characteristics of the air flowing through the insert 102.

With reference to FIGS. 21, 22 and 23, another embodiment of a sleep surface system 650 is provided. In this embodiment, the sleep surface system 650 may be sized and configured to be associated or incorporated with a head pillow (not shown) for a person. For example, in one embodiment, the sleep surface system 650 may include an outer liner 652, a forced air device 654, and a plenum 656. The forced air device 654 may be employed to force air into the outer liner 652 with the plenum 656 disposed within the outer liner 652 to assist in distributing the air within the outer liner 652.

The outer liner 652 may also be referenced as a pillow case member herein. The outer liner 652 may extend with a sleeve like structure with one end 658 permanently closed and the other opposite side end 660 may be moveable between an open position and a closed position. In the open position, the outer liner 652 may receive a head pillow (not shown) or the head pillow may be removed from the outer liner 652. In the use position of the sleep surface system 650, the outer liner 652 may be moved to a closed position with, for example, a zipper structure 664 or any other structure that may be employed for closing the side end 660 so that the outer liner 652 defines an enclosed space. The outer liner 652 may also include a port opening 662, similar to previous embodiments, for coupling, for example, a fan output 666 of the forced air device 654 to the port opening 662 of the outer liner 652 to facilitate forcing air within the outer liner 652. The port opening 662 may be positioned adjacent to one end of the zipper structure 664 or any other suitable location adjacent a periphery 668 of the outer liner 652.

The outer liner 652 may be formed of a woven material 670, similar to the top liner 606 of the sleep surface system 600 of the previous embodiment (FIG. 20), such that the material may be formed from woven fibers, natural or synthetic, or a blend thereof. The woven material 670 may exhibit a weave density or weave tightness so as to define spaces or pores 672 therein in the material itself that facilitate air forced within the outer liner 652 or pillow case member to provide air flow through the pores 672 to an exterior surface 674 of the outer liner 652, as shown by arrows 676 (FIG. 23).

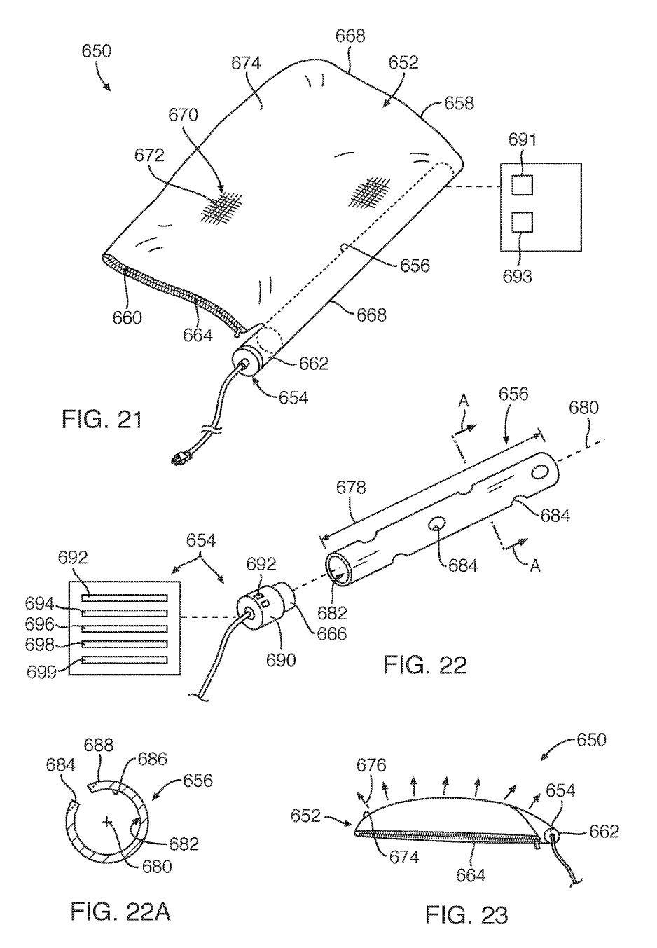

Now with reference to FIGS. 21, 22 and 22A, the sleep surface system 650 of this embodiment may include the before-identified plenum 656. The plenum 656 may be coupled to the fan output 666 of the forced air device 654 and positioned adjacent the periphery 668 of the outer liner 652 and within the outer liner 652 so as to be positioned alongside a head pillow (not shown) also positioned within the outer liner 652. Further, the plenum 656 may be readily removable and disassembled from the outer liner 652 and the forced air device 654 to facilitate washing the outer liner as desired.

In one embodiment, the plenum 656 may be elongated with a length 678 to extend along a longitudinal axis 680. In another embodiment, the plenum 656 may be an elongated tubular structure. The plenum 656 may be flexible and formed of a resilient material. In one embodiment, the plenum 656 may be a foam material. The plenum may include a hollow portion 682, such as a bore extending along the longitudinal axis 680, with multiple openings 684 defined therein and along the length 678 of the plenum 656. The multiple openings 684 may extend from an internal surface 686 of the hollow portion 682 to an exterior surface 688 of the plenum 656 so that air can flow into the hollow portion 682 of the plenum 656 and be distributed from the multiple openings 684 defined in the plenum 656. The multiple openings 684 defined in the plenum 656 may be at separate and distinct locations of the plenum 656, such as along the length 678 of the plenum 656. The multiple openings 684 may be along opposite sides of the plenum 656 in a symmetrical manner and/or at random locations along the plenum 656. As such, the multiple openings 684 may facilitate the distribution of the air into the outer liner 652. In this manner, should some of the openings 684 become inadvertently blocked with, for example, the arm of a person laying on the pillow case member or outer liner 652 the other non-blocked openings 684 may distribute the air within the outer liner 652 and out the pores 672 not being blocked another surface, such as the surface the outer liner 652 is positioned upon.

Similar to previous embodiments, the forced air device 654 may include a blower fan 690 and manual controls 692 for fan speed and/or switching the fan to an on position and off position. Further, similar to previous embodiments, the forced air device 654 or sleep surface system 600 may include a filter 692, such as a Hepa filter or the like (as shown in schematic form). Such filter 692 may be positioned over the path of the air flow, such as being directly coupled to the blower fan 690 or over the length 678 of the plenum 656. Further, as shown in schematic form in FIG. 22, in another embodiment, the forced air device 654 may include one or more sensors and/or controllers, such as a temperature sensor 694 and a controller 696 associated with a thermal element 698, such as a cooling element or heating element, or any other sensor 699 that may be embedded within the sleep surface system 650, as described herein in previous embodiments. With this arrangement, the filter 692 associated with the forced air device 654 or sleep surface system 600 may provide clean air flow within the outer liner 652 or enclosed pillow case member to then flow through the pores 672 defined in the woven material 670 of the outer liner 652 or pillow case member. Such clean air flow flowing through the pores 672 of the outer liner 652 may be useful to a person sleeping thereon who may be suffering from allergies or the like. Further, maintaining air flow through the pores 672 of the outer liner 652 may be desirable for keeping the exterior surface 674 of the outer liner 652 feeling fresh and cool to the user.

As depicted in FIG. 21, in another embodiment, the sleep surface system may include a sensor 691 and a vibrator/speaker 693 associated with the outer liner 652 or plenum 656. The sensor 691 may be configured to sense snoring of the person laying thereon. Upon sensing snoring in the person, the vibrator/speaker 693 may be activated to vibrate or make noise to jostle or slightly wake the person until the person, for example, adjusts his/her sleeping position and stops snoring.

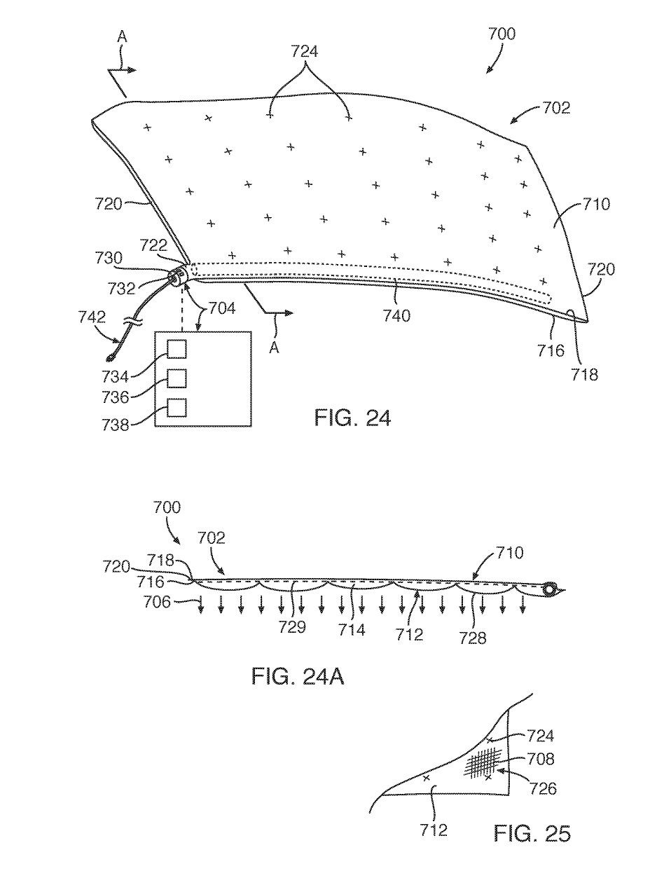

Now with reference to FIGS. 24, 24A, and 25, another embodiment of a sleep surface system 700 is provided. In this embodiment, the sleep surface system 700 may include a blanket member 702 and a forced air device 704 such that the blanket member 702 may be positioned over a person to provide generally downward air flow, as depicted by arrows 706, through pores 708 defined in the blanket member 702. For example, the blanket member 702 may include a top liner 710 coupled to a bottom liner 712 with a space 714 defined between the top and bottom liners 710, 712. The bottom liner 712 may be coupled to the top liner 710 with stitching along or adjacent to a bottom liner periphery 716. Similarly, the top liner 710 may be coupled to the bottom liner 712 along or adjacent to a top liner periphery 718. As such, the peripheries of the top and bottom liner 710, 712 may include a generally common periphery 720 of the blanket member 702 that may include a square or rectangular shape, for example. The blanket member 702 may also include a port opening 722 adjacent the periphery 720 of the blanket member 702, such as adjacent one of the corners of the blanket member 702, the port opening 722 providing air flow communication to the space 714 defined in the blanket member 702. Similar to previous embodiments, the port opening 722 may be sized and configured to receive an output portion of the forced air device 704 to force air into the space 714 between the top and bottom liners 710, 712. Further, the top liner 710 may be coupled to the bottom liner 712 at multiple separate and discrete attachment locations 724 that may be separate from peripheral stitching or bonding between the top and bottom liners 710, 712 or the periphery 720 of the blanket member 702. Such attachment locations 724 may be employed with, for example, one or more filaments, at each of the separate and discrete attachment locations 724 in a stitched or knotted manner, to form point or line type attachments or the like. The top liner 710 may be formed with a non-porous or impermeable material that substantially prevents air flow through the material of the top liner 710. In this embodiment, the bottom liner 712 may be formed of a woven material 726 with a density or woven tightness to provide pores 708 within the woven material 726 itself, similar to the previous embodiments. In this manner, the bottom liner 712 with the pores 708 defined therein may facilitate air flow, as indicated by arrows 706, from the space 714 defined in the blanket member 702 to an exterior surface 728 of the bottom liner 712.

In another embodiment, the blanket member 702 may include batting 729 (as depicted with dashed lines of FIG. 24A) positioned within the space 714 between the top and bottom liners 710, 712. In another embodiment, the bottom liner 712 may be coupled to the batting 729 at multiple separate and discrete attachment locations with one or more filaments in a knotted manner or the like (similar to that described relative to the top and bottom liners 710, 712). With such arrangement, the top liner 710 may also be coupled to the batting 729 at multiple separate and discrete attachment locations. Alternatively, the top liner 710 may be attached to the batting 729 with an adhesive or other bonding mechanism extending, for example, over non-discrete portions or substantially an entire surface area of the inner surface of the top liner 710.

In one embodiment, the forced air device 704 of the sleep surface system 700 may include a blower fan 730 and manual controls 732. Further, as depicted partially in schematic form, the forced air device 704 of the sleep surface system 700 may include a thermal element 734, a temperature sensor 736 and a controller 738, similar to that depicted and described in previous embodiments. With this arrangement, a desired temperature for air flow through the blanket member 702 may be selected, controlled and maintained with the thermal element 734 (with a heating element or a cooling element) associated with the forced air device 704. For example, a user or other person may select a desired temperature for the air moving through the bottom liner 712 of the blanket member 702, the temperature sensor 736 may sense the actual temperature of the air being blown from the blower fan 730, and the thermal element 734 may be activated to change the temperature of the air being blown to substantially correspond to the desired temperature selected. In this manner, the blanket member 702 of the sleep surface system 700 may be positioned over a user to facilitate warming or cooling of the user at the selected temperature. Such sleep surface system 700 with the blanket member 702 may be useful in a hospital setting as well for common home and recreational use.

In another embodiment, the blanket member 702 and sleep surface system 700 may incorporate a plenum 740 (as depicted in dashed lines in FIG. 24), similar to that described in the sleep surface system 650 of the previous embodiment (see FIGS. 21 and 22). The plenum 740 may be coupled to an output portion (not shown) of the forced air device 704 and removably positioned within the space 714 between the top and bottom liners 710, 712 and along or adjacent a periphery 720 of the blanket member 702. For example, the plenum 740 may be elongated and tubular with multiple openings, the openings sized and positioned along the length of the plenum so as to distribute the air from the plenum 740 to fill the space defined in the blanket member 702 to then move through the pores 708 in a generally downward direction (as depicted by arrows 706) from the bottom liner 712.

The forced air device 704 of this embodiment (and the other embodiments described herein) may be coupled to and powered directly from an electrical outlet with a typical cord and plug arrangement 742. In another embodiment, the forced air device 704 of this and other embodiments described herein may include, for example, a rechargeable battery pack coupled thereto or incorporated therewith that may be charged during non-use of the forced air device 704. The forced air device 704 may then later be operated from the stored energy of the charged battery pack without the electrical cord connected thereto.

Further, the plenum employed with the sleep surface systems of FIGS. 21 and 24, may also be employed with the sleep surface system 600 sized for an infant's mattress, the plenum 744 depicted in dashed lines, as shown in FIG. 20. The plenum 744 that may be incorporated in the sleep surface system of FIG. 20 in a readily removable manner that may be removably coupled to the forced air device 604 and may be positioned within the space 612 defined between the top and bottom liners 606, 608 and along or adjacent the periphery 610 of the top liner 606. Such plenum 744 may include similar structure and features as that depicted in FIG. 22 and may be sized and configured to distribute air within the space 612 without being inadvertently blocked, as previously set forth.

While the invention may be susceptible to various modifications and alternative forms, specific embodiments have been shown by way of example in the drawings and have been described in detail herein. Further, the structural features of any one embodiment disclosed herein may be combined or replaced by any one of the structural features of another embodiment set forth herein. As such, it should be understood that the invention is not intended to be limited to the particular forms disclosed. Rather, the invention includes all modifications, equivalents, and alternatives falling within the spirit and scope of the invention as defined by the following appended claims.

What is claimed is:

1. A sleep surface system for positioning under an infant on a sleep surface, the sleep surface system comprising:
a removable insert configured to be removably positioned over the sleep surface, the removable insert including a port opening extending to a substantially enclosed space defined between a top liner and a bottom liner with a peripheral boundary defining a coupling between the top and bottom liners, the top liner having an exterior surface and being formed of a material with pores defined therein such that the pores extend between the enclosed space and the exterior surface of the top liner, the top liner being coupled to the bottom liner at multiple discrete locations positioned separate from the peripheral boundary between the top liner and the bottom liner; and
a forced air device configured to provide air flow into the enclosed space defined in the removable insert, the air flow configured to flow into the enclosed space defined within the removable insert and out of the pores defined therein.

2. The sleep surface system of claim 1, wherein the removable insert comprises a peripheral portion extending from the peripheral boundary sized and configured to extend to an underside of the sleep surface.

3. The sleep surface system of claim 1, wherein the insert comprises an elastic portion sized and configured to temporarily couple the insert to the sleep surface.

4. The sleep surface system of claim 1, wherein the port opening is configured to facilitate the air flow from the forced air device to the substantially enclosed space defined in the removable insert.

5. The sleep surface system of claim 1, further comprising an elongated plenum configured to be positioned adjacent the peripheral boundary between the top and bottom liners and within the space of the removable insert, the plenum including openings along a length of the elongated plenum for distributing air within the enclosed space of the removable insert.

6. The sleep surface system of claim 1, wherein the bottom liner is a non-porous material.

7. A sleep surface system for use with a head pillow, the sleep surface system comprising:
a pillow case member including a liner with an inner surface and an outer surface, the pillow case member defining an enclosable space within the pillow case member such that the space is sized and configured to receive the head pillow, the pillow case member including one side end configured to be positioned between an open position and a closed position, the pillow case member including a port opening defined therein, the pillow case member including a material defining multiple pores therein;
a forced air device configured to provide air flow into the enclosable space defined in the pillow case member through the channel port of the pillow case member, the air flow configured to flow through the port opening and into the enclosable space and out of the multiple pores defined in the liner; and
an elongated plenum coupled to the forced air device and positionable adjacent a periphery of the pillow case member and within the space defined in the pillow case member, the elongated plenum including openings along a length of the elongated plenum configured to distribute air within the space of the pillow case member.

8. The sleep surface system of claim 7, wherein the liner comprises an upper liner and a lower liner with the spaced defined therebetween, the upper liner including the multiple pores defined therein to facilitate air flow therethrough.

9. The sleep surface system of claim 8, wherein the lower liner comprises a permeable material so as to facilitate air flow through pores of the lower liner.

10. The sleep surface system of claim 7, wherein the plenum comprises a tubular structure.

11. The sleep surface system of claim 7, wherein the plenum comprises a foam material.

12. The sleep surface system of claim 7, wherein the plenum comprises one end coupled to an output housing of the forced air device.

13. The sleep surface system of claim 7, wherein the forced air device is positionable at least partially within the port opening of the pillow case member.

14. A blanket system for positioning over a person, the blanket system comprising:
a blanket member configured to be removably positioned over the person, the blanket member including a top liner and a bottom liner and a port opening, the top liner and bottom liner defining a space therebetween, the top liner being coupled to the bottom liner at multiple discrete locations separate from a periphery of the top and bottom liners, the bottom liner formed of a material with pores defined therein, the pores extending between the space and an outer surface of the bottom liner so as to facilitate air flow therethrough; and
a forced air device configured to provide air flow through the port opening and into the space defined in the blanket member so as to flow air through the pores of the bottom liner.

15. The blanket system of claim 14, wherein the forced air device comprises a heating element associated therewith to force heated air through the pores of the blanket member.

16. The blanket system of claim 14, wherein the forced air device comprises a cooling element associated therewith to force cooled air through the pores of the blanket member.

17. The blanket system of claim 14, further comprising a plenum coupled to the forced air device and positioned adjacent the periphery of the blanket member and within the space of the blanket member.

18. The blanket system of claim 17, wherein the plenum comprises multiple openings along a length of the plenum to distribute air therethrough.

19. The blanket system of claim 14, wherein the blanket member comprises batting within the space between the top and bottom liners.

20. The blanket system of claim 14, wherein the top liner comprises an impermeable liner.
